(19) United States
(12) Patent Application Publication
(10) Pub. No.: US 2013/0209549 A1
(43) Pub. Date: Aug. 15, 2013

(54) MATERIALS AND METHODS FOR TREATING NEURODEGENERATIVE DISEASES

(71) Applicant: Chad Dickey, Tampa, FL (US)

(72) Inventor: Chad Dickey, Tampa, FL (US)

(73) Assignee: UNIVERSITY OF SOUTH FLORIDA, TAMPA, FL (US)

(21) Appl. No.: 13/809,537

(22) PCT Filed: Jul. 21, 2011
(86) PCT No.: PCT/US2011/044885

§ 371 Date: Apr. 29, 2013

Related U.S. Application Data
(60) Provisional application No. 61/400,042, filed on Jul. 21, 2010.

(51) Int. Cl. A61K 38/17; A61K 45/06; A61K 31/708

(52) U.S. Cl. CPC A61K 38/17; A61K 31/708; A61K 45/06
USPC 424/450; 514/17.7; 514/17.8; 514/44 R

(57) ABSTRACT

The subject invention concerns materials and methods for treating neurodegenerative diseases and conditions associated with aggregation of the microtubule-associated protein tau. A method of the invention comprises administering an effective amount of a heat shock protein 27 (Hsp27), or a biologically-active fragment or variant thereof, or a polynucleotide encoding the same, to a person or animal in need of treatment. In one embodiment, a heat shock protein 27 (Hsp27), or a biologically-active fragment or variant thereof, is contacted with or provided to a target cell. The target cell can be a neuron. In a specific embodiment, an Hsp27 is delivered to the target cell via a polynucleotide encoding an Hsp27 protein, or a biologically-active fragment or variant thereof. In one embodiment, a method of the invention comprises injecting (e.g., via stereotaxic injection) a polynucleotide expression construct of the invention comprising a polynucleotide encoding an Hsp27 protein directly into neural tissue of a person or animal. The subject invention also concerns compositions comprising i) a heat shock protein 27, or a biologically-active fragment or variant thereof, and/or ii) a polynucleotide encoding an Hsp27, or a biologically-active fragment or variant thereof. The polynucleotide can be an expression construct that provides for expression of an Hsp27 in a target cell.

### CROSS-REFERENCE TO RELATED APPLICATION

The present application claims the benefit of U.S. Provisional Application Ser. No. 61/400,042, filed Jul. 21, 2010, which is hereby incorporated by reference herein in its entirety, including any figures, tables, nucleic acid sequences, amino acid sequences, or drawings.

### GOVERNMENT SUPPORT

This invention was made with government support under grant number R00AG031291 awarded by the National Institute on Aging. The government has certain rights in the invention.

### BACKGROUND OF THE INVENTION

Aberrant protein production is a common feature of many diseases of aging. The majority of these disease-associated proteins are also clients of the chaperone network. These abnormal clients are prone to aggregation, forming pathologic inclusion bodies in neurodegenerative diseases. One family of chaperones that may help offset the toxicity of misfolded substrates is the small heat shock protein (Hsp) family. The primary function of small Hsps is to protect unfolded proteins in the cytosol from entering an aggregation pathway. This function has been defined primarily by investigating one particular small Hsp: Hsp27 (Renkawek et al. 1999; Wyttenbach et al. 2002; Shimura et al. 2004; Sanbe et al. 2007). Hsp27 regulates many disease-related proteins that are prone to aggregation; however, validation of these findings in mammalian systems has been slow to follow, due in large part to several unique properties that distinguish Hsp27 from more classical chaperones like Hsp70 and Hsp90 (Perrin et al. 2007).

Hsp27 facilitates degradation and prevents aggregation of aberrant substrates independent of ATP or ubiquitination (Jakob et al. 1993; Shimura et al. 2004). Thus, measuring its activity is difficult since it essentially has no measurable enzymatic function. The most well-characterized modification of Hsp27 is its capacity to be phosphorylated by stress-activated kinases at serine residues 15, 78, and 82 (Stokoe et al. 1992). The known consequence of this phosphorylation is disassembly of large (200 kDa-800 kDa) dormant Hsp27 multimers into smaller oligomeric and monomeric species (Huot et al. 1991; Landry et al. 1992). These structures are not static, and deciphering the role that cycling between phosphorylated and dephosphorylated states has on chaperoning activity will be a critical step forward in understanding the mechanisms used by Hsp27 to process substrates (Haley et al. 2000; Lelj-Garolla and Mauk 2005; Lelj-Garolla and Mauk 2006).

Our previous work has shown that regulation of the microtubule associated protein tau, which accumulates in ˜70% of all neurodegenerative diseases, is tightly coupled to the chaperone machinery. While a role for Hsp27 in tau regulation has been proposed (Shimura et al. 2004), its involvement with tau in vivo is unknown. Neurons normally have low levels of Hsp27 expression, but this can be induced in response to proteotoxic stress. In fact, increases in Hsp27 correlate with pathological accumulation of aberrant proteins in neurodegenerative diseases, both in neurons and in glia (Renkawek et al. 1994; Koren et al. 2009).

### BRIEF SUMMARY OF THE INVENTION

The subject invention concerns materials and methods for treating neurodegenerative diseases and conditions associated with aggregation of the microtubule-associated protein tau. A method of the invention comprises administering an effective amount of a heat shock protein 27 (Hsp27), or a biologically-active fragment or variant thereof, or a polynucleotide encoding the same, to a person or animal in need of treatment. In one embodiment, a heat shock protein 27 (Hsp27), or a biologically-active fragment or variant thereof, is contacted with, or delivered or provided to a target cell. The target cell can be a neuron. In a specific embodiment, an Hsp27 is delivered to the target cell via a polynucleotide encoding an Hsp27 protein, or a biologically-active fragment or variant thereof. In one embodiment, the Hsp27 protein is a human Hsp27. In a specific embodiment, the human Hsp27 protein comprises the amino acid sequence of SEQ ID NO:1 (GenBank Accession No. NM_{—}001540), or a biologically-active fragment or variant thereof. In a more specific embodiment, the human Hsp27 protein is encoded by a polynucleotide comprising the nucleotide sequence of SEQ ID NO:2 (GenBank Accession No. NM_{—}001540). In one embodiment, a method of the invention comprises injecting (e.g., via stereotaxic injection) a polynucleotide expression construct of the invention comprising a polynucleotide encoding an Hsp27 protein directly into neural tissue of a person or animal. In a specific embodiment, the expression construct is an adeno-associated viral construct. An expression construct of the invention can be designed to provide for constitutive expression of an Hsp27 protein in a target cell or tissue.

The subject invention also concerns compositions comprising i) a heat shock protein 27, or a biologically-active fragment or variant thereof, and/or ii) a polynucleotide encoding an Hsp27, or a biologically-active fragment or variant thereof. The polynucleotide can be an expression construct that provides for expression of an Hsp27 in a target cell. In one embodiment, the Hsp27 is a human Hsp27. In a specific embodiment, the human Hsp27 protein comprises the amino acid sequence of SEQ ID NO:1, or a biologically-active fragment or variant thereof. In a more specific embodiment, the human Hsp27 protein is encoded by a polynucleotide comprising the nucleotide sequence of SEQ ID NO:2.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B. Wt and 3×S/D Hsp27 bind tau despite differences in their oligomerization states. (FIG. 1A) SC-WB for Hsp27. Recombinant wt and 3×S/D Hsp27 were independently subjected to SC ultracentrifugation. Hsp27 levels were analyzed in equal volumes of the recovered SC fractions. (FIG. 1B) CoIP-WB of his-tagged recombinant tau (rTau) with rabbit anti-tau antibody showed that rTau co-precipitated with both wt and 3×S/D Hsp27, but not BSA. Unlabelled rabbit IgG was used as a negative co-IP control. Sucrose cushion separation of tau pre-incubated alone or with either wtHsp27 or 3×S/D Hsp27. FIGS. 2A-2D. Heparin-induced tau fibril formation is abrogated by wtHsp27 and 3×S/D Hsp27. (FIG. 2A) Tau (circles) fibril growth rate in the absence or presence of either wtHsp27 (squares) or 3×S/D Hsp27 (triangles). (FIG. 2B and FIG. 2C) Particle size monitoring of tau alone (circles) or in combination with wt (squares) or 3×S/D (triangles) Hsp27 at day 0 (FIG. 2B) and day 5 (FIG. 2C) by DLS. (FIG. 2D) AFM images of tau alone or pre-incubated with either wtHsp27 or 3×S/D Hsp27. Buffer without protein was used as a negative control. Heparin was used to induce tau aggregation for 5 days (FIGS. 2A, 2B, and 2C), and 15 days (FIG. 2D). Pre-incubation consisted of 35 moles of recombinant tau per mole of recombinant Hsp27s.

FIGS. 3A and 3B. Hsp27 up-regulation by intra-hippocampal AAV9 injections has robust distribution throughout the hippocampus, but it does not transduce all neurons. (FIG. 3A) IHC detection of Hsp27 in brain sections of mice injected with AAV1, AAV9, or non-injected mice. Brains were harvested eight weeks after viral injections. Dark staining, which is not present in the non-injected brain section, corresponds to Hsp27. (FIG. 3B) Immuno-fluorescence of the hippocampus neuronal layer using antibodies against Hsp27 and the neuronal marker NeuN shows that not all neurons (blue) expressed Hsp27 (green).

FIGS. 4A-4C. Neuronal over-expression of Hsp27-AAV9 variants differentially modulates neuronal tau levels. (FIG. 4A) Confocal images of immuno-fluorescently labeled neurons (blue), tau (red), and Hsp27 or GFP (green) in CA1 from rTg4510 mice injected with AAV9-expressing wtHsp27, 3×S/D Hsp27, or GFP (scale bar=125 μm). (FIG. 4B) Higher zoom images of fields from CA1 regions of IF-labeled sections from AAV9-injected mice (scale bar=50 μm). (FIG. 4C) Quantification of red signal (tau) in blue areas (neurons) sharing positive green signal (Hsp27 or GFP; see supplement for further details). Compared to GFP-expressing neurons (n=264), tau signal was reduced by 66% (n=296) and increased by 50% in neurons expressing wtHsp27 and 3×S/D Hsp27 (n=302), respectively (p<0.001). At least 3 sections were analyzed for each group of mice. Number of mice: n_{wtHsp27}=6, n_{3×S/D}=5, n_{GFP}=4. Statistical significance was determined with Student T-tests.

FIGS. 5A and 5B. WtHsp27 is unable to disrupt pre-formed tau aggregates. (FIG. 5A) DLS scatter plot measuring the particle size of stably-formed recombinant tau fibrils before and after addition of wtHsp27. Heparin was used to induce aggregation over the course of 22 days. WtHsp27 was then incubated and particle size was measured over time. (FIG. 5B) Tau can enter 3 protein folding pathways once it loses form and function: aggregation (Kagg), degradation (degrade) or refolding (Krefold). Aggregation and refolding of tau is based on equilibrium, as indicated by the bi-directional arrows. Hsp27 prevents tau aggregation and facilitates the entry of tau into degradation or refolding pathways. Since refolding, or recycling of tau is in equilibrium with unfolded intermediates, it is possible that Hsp27 may increase the prevalence of these intermediates. Hsp27 is not able to disaggregate tau once fibrillization has already occurred.

FIGS. 6A and 6B. LTP deficits in rTg4510 mice are rescued by wtHsp27, but remain unaffected with 3×S/D Hsp27. After recording baselines for 20 mM, LTP was induced with TBS (5 bursts of 200 Hz separated by 200 ms, repeated 6 times with 10 s between the 6 trains) and LTP was recorded for 60 min Changes in fEPSP slope are expressed as a percentage of baseline. (FIG. 6A) Representative fEPSP traces for rTg4510 and age-matched NTG mice injected with either saline or GFP-AAV9. Changes in the slopes of NTG-GFP (n=6) and NTG-Saline (n=9) were significantly different (*p<0.01) from that of TG-GFP (n=5) and TG-Saline (n=9). LTP was not significantly different between mice of the same genotype despite treatments (p>0.05). (FIG. 6B) Representative fEPSP traces for rTg4510 injected with wtHsp27-AAV9 (n=7), 3×S/D-AAV9 Hsp27 (n=9), or GFP-AAV9 (7), and age-matched NTG mice injected with GFP-AAV9 (n=6). Changes in fEPSP slopes between NTG-GFP vs. TG-wtHsp27 and TG-3×S/D vs. TG-GFP were not significantly different (p>0.05). NTG-GFP and TG-wtHsp27 fEPSP slopes were significantly different from both TG-3×S/D and TG-GFP (p<0.001). P-values were determined by student T-tests using data from the first and last 10 min of the LTP curves.

FIG. 7. Proposed mechanism of wtHsp27 and 3×S/D Hsp27 modulation of tau aggregation. Upon stress-induced client denaturation or loss of function, both wtHsp27 and phospho-Hsp27 (S/DHsp27) bind and prevent client aggregation (in vitro). Client-bound wtHsp27 is then able to facilitate client clearance. Conversely, retention of Hsp27 in a mock-phosphorylated state subverts client clearance, leading to accumulation of soluble intermediates (in vivo).

FIGS. 8A and 8B. Tau levels are decreased in hippocampus of tau transgenic mice injected with AAV9-wtHsp27. WB of hippocampal lysates from transgenic mice injected with either AAV9-GFP or -wtHsp27. Compared to GFP-injected hippocampi, wtHsp27-expressing tissue had an 81% reduction in tau levels (*p<0.05). Statistical significance was established by Student T-test.

FIG. 9. Input-output graph for LTP experiments. Output field analysis following increases of field stimulation in 0.5 mV increments until 15 mV. Data were fit with non-linear regression and showed no significant difference.

FIGS. 10A-10J. Microscopy analysis. Quantification of the tau signal in neurons expressing Hsp27 (wt or 3×S/D) or GFP was done as follows: the totality of the signals from z-stacked images for each ROI was summed into a final image that was imported from the Leica software into ImageJ (FIG. 8A). The three channels were split into independent images (FIGS. 10B-10D). To obtain a mask of NeuN-positive regions, the blue channel was processed by removing outliers (Process>Noise>Remove Outliers>radius 10 pixels, threshold of 5, and remove Bright). This image was then thresholded to a grayscale range of 70-255. We selected the Area and the Integrated Density (Analyze>Set Measurements) and redirected to the red and green channels. The following changes were adjusted in Analyze>Analyze Particles: we further specified our NeuN-specific ROI to areas greater that 100 pixels² and circularity of 0.00-1.00, which increased the selectivity of our measurements to neurons. We also excluded areas along the edges of the field. Since the neuron somas were sometimes dissected by the z-stack, we included the area within the limits of blue delineations, which correspond to areas where neuron bodies are present.

### BRIEF DESCRIPTION OF THE SEQUENCES

SEQ ID NO:1 is an amino acid sequence of a human Hsp27 polypeptide.

SEQ ID NO:2 is a polynucleotide sequence encoding a human Hsp27 polypeptide having the amino acid sequence of SEQ ID NO:1.

### DETAILED DESCRIPTION OF THE INVENTION

The subject invention concerns materials and methods for treating or preventing neurodegenerative diseases and conditions associated with aggregation of the microtubule-associated protein tau. Recombinant wildtype Hsp27 and a genetically altered version of Hsp27 that is perpetually pseudo-phosphorylated (3×S/D) were generated. Both Hsp27 variants interacted with tau, and atomic force microscopy and dynamic light scattering showed that both variants could also prevented tau filament formation. However, extrinsic genetic delivery of these two Hsp27 variants to tau transgenic mice using adeno-associated viral particles showed that wildtype Hsp27 reduced neuronal tau levels, while 3×S/D Hsp27 was associated with increased tau levels. Moreover, rapid decay in hippocampal long-term potentiation (LTP) intrinsic to this tau transgenic model was rescued by wildtype Hsp27 over-expression, but not by 3×S/D Hsp27. Since the 3×S/D Hsp27 mutant cannot cycle between phosphorylated and de-phosphorylated states, it appears that Hsp27 must be functionally dynamic to facilitate tau clearance from the brain and rescue LTP; however when this property is compromised, Hsp27 may actually facilitate accumulation of soluble tau intermediates.

In one embodiment, a method of the invention for treating or preventing a neurodegenerative disease or a condition associated with aggregation of tau protein comprises administering an effective amount of a heat shock protein 27 (Hsp27), or a biologically-active fragment or variant thereof, or a polynucleotide encoding the same, to a person or animal in need of treatment. In a specific embodiment, the Hsp27 protein is capable of being in phosphorylated and non-phosphorylated forms, e.g., the Hsp27 protein can cycle between being in a phosphorylated form and being in a non-phosphorylated form. In one embodiment, the disease or condition is characterized by the presence of neurofibrillary tangles of protein tau and/or hyperphosphorylated protein tau in a cell, such as a neuronal or glial cell. Diseases and conditions contemplated within the scope of the invention include, but are not limited to, Alzheimer's disease, gangliogliomas and gangliocytomas, argyrophilic grain dementia, corticobasal degeneration, dementia pugilistica, frontotemporal dementia with parkinsonism linked to chromosome17, Pick's disease, Hallervorden-Spatz disease, myotonic dystrophy, Niemann-Pick disease (type C), Parkinsonism-dementia complex of Guam, postencephalitic parkinsonism, prion diseases (some), progressive subcortical gliosis, and progressive supranuclear palsy. In a specific embodiment of the method, an Hsp27 is administered to the person or animal or is delivered to a target cell in the person or animal via a polynucleotide encoding an Hsp27 protein, or a biologically-active fragment or variant thereof. In one embodiment, the Hsp27 protein is a human Hsp27 (GenBank Accession No. NM_{—}001540; Swiss-Prot P04792). In a specific embodiment, the human Hsp27 protein comprises the amino acid sequence of SEQ ID NO:1, or a biologically-active fragment or variant thereof. In a more specific embodiment, the human Hsp27 protein is encoded by a polynucleotide comprising the nucleotide sequence of SEQ ID NO:2. In one embodiment, a method of the invention comprises injecting (e.g., via stereotaxic injection) a polynucleotide expression construct of the invention comprising a polynucleotide encoding an Hsp27 protein directly into neural tissue and/or cells of a person or animal. In a specific embodiment, the expression construct is an adeno-associated viral construct. Optionally, other drugs and therapeutics used in treating neurodegenerative diseases and conditions can also be administered to the human or animal. The other drugs and therapeutics can be administered to the human or animal prior to, at the same time (co-administered), or subsequent to administration of the Hsp27 protein or polynucleotide encoding the same. Examples of other drugs or therapeutics that can be administered include, but are not limited to, donepezil (Aricept), galantamine (Razadyne), rivastigmine (Exelon), memantine (Akatinol), and L-dopa.

The subject invention also concerns methods for improving memory function in a human or animal. In one embodiment, a method of the invention comprises administering an effective amount of a heat shock protein 27 (Hsp27), or a biologically-active fragment or variant thereof, or a polynucleotide encoding the same, to a person or animal in need of treatment. In a specific embodiment, the Hsp27 protein is capable of being in phosphorylated and non-phosphorylated forms. In a specific embodiment of the method, an Hsp27 is administered to the person or animal or is delivered to a target cell in the person or animal via a polynucleotide encoding an Hsp27 protein, or a biologically-active fragment or variant thereof. In one embodiment, the Hsp27 protein is a human Hsp27 (GenBank Accession No. NM_{—}001540; Swiss-Prot P04792). In a specific embodiment, the human Hsp27 protein comprises the amino acid sequence of SEQ ID NO:1, or a biologically-active fragment or variant thereof. In a more specific embodiment, the human Hsp27 protein is encoded by a polynucleotide comprising the nucleotide sequence of SEQ ID NO:2. In one embodiment, a method of the invention comprises injecting (e.g., via stereotaxic injection) a polynucleotide expression construct of the invention comprising a polynucleotide encoding an Hsp27 protein directly into neural tissue and/or cells of a person or animal. In a specific embodiment, the expression construct is an adeno-associated viral construct. Optionally, other drugs and therapeutics used in improving memory function can also be administered to the human or animal. The other drugs and therapeutics can be administered to the human or animal prior to, at the same time (co-administered), or subsequent to administration of the Hsp27 protein or polynucleotide encoding the same. Examples of other drugs or therapeutics that can be administered include, but are not limited to, donepezil (Aricept), galantamine (Razadyne), rivastigmine (Exelon), and memantine (Akatinol).

The subject invention also concerns methods for decreasing the levels of the protein tau in a target cell. In one embodiment, a heat shock protein 27 (Hsp27), or a biologically-active fragment or variant thereof, or a polynucleotide encoding the same, is contacted with or provided to a target cell. The target cell can be a neuron or a glial cell. In a specific embodiment, the Hsp27 protein is capable of being in phosphorylated and non-phosphorylated forms. In a specific embodiment, an Hsp27 is delivered to the target cell via a polynucleotide encoding an Hsp27 protein, or a biologically-active fragment or variant thereof. In one embodiment, the Hsp27 protein is a human Hsp27. In a specific embodiment, the human Hsp27 protein comprises the amino acid sequence of SEQ ID NO:1, or a biologically-active fragment or variant thereof. In a more specific embodiment, the human Hsp27 protein is encoded by a polynucleotide comprising the nucleotide sequence of SEQ ID NO:2. In one embodiment, a method of the invention comprises injecting (e.g., via stereotaxic injection) a polynucleotide expression construct of the invention comprising a polynucleotide encoding an Hsp27 protein directly into neural tissue or cells of a person or animal. In a specific embodiment, the expression construct is an adeno-associated viral construct. An expression construct of the invention can be designed to provide for constitutive expression of an Hsp27 protein in a target cell or tissue. Optionally, other drugs and therapeutics used in methods for decreasing the levels of the protein tau in a target cell can also be contacted or provided to a target cell. The other drugs and therapeutics can be provided or contacted prior to, at the same time (co-administered), or subsequent to administration of the Hsp27 protein or polynucleotide encoding the same. Examples of other drugs or therapeutics that can be administered include, but are not limited to, paclitaxel, a statin, lithium, inhibitors of cyclin-dependent kinase 5, and methylthionine chloride.

The subject invention also concerns methods for treating and/or preventing hippocampal long-term potentiation decay or impairment in a person or animal. In one embodiment, a method of the invention comprises administering an effective amount of a heat shock protein 27 (Hsp27), or a biologically-active fragment or variant thereof, or a polynucleotide encoding the same, to a person or animal in need of treatment. In a specific embodiment, the Hsp27 protein is capable of being in phosphorylated and non-phosphorylated forms. In a specific embodiment of the method, an Hsp27 is administered to the person or animal or is delivered to a target cell in the person or animal via a polynucleotide encoding an Hsp27 protein, or a biologically-active fragment or variant thereof. In one embodiment, the Hsp27 protein is a human Hsp27 (GenBank Accession No. NM_{—}001540; Swiss-Prot P04792). In a specific embodiment, the human Hsp27 protein comprises the amino acid sequence of SEQ ID NO:1, or a biologically-active fragment or variant thereof. In a more specific embodiment, the human Hsp27 protein is encoded by a polynucleotide comprising the nucleotide sequence of SEQ ID NO:2. In one embodiment, a method of the invention comprises injecting (e.g., via stereotaxic injection) a polynucleotide expression construct of the invention comprising a polynucleotide encoding an Hsp27 protein directly into neural tissue and/or cells of a person or animal. In a specific embodiment, the expression construct is an adeno-associated viral construct. Optionally, other drugs and therapeutics used in methods for treating and/or preventing hippocampal long-term potentiation decay or impairment in a person or animal can also be administered to the human or animal. The other drugs and therapeutics can be administered to the human or animal prior to, at the same time (co-administered), or subsequent to administration of the Hsp27 protein or polynucleotide encoding the same.

The subject invention also concerns compositions comprising i) a heat shock protein 27, or a biologically-active fragment or variant thereof, and/or ii) a polynucleotide encoding an Hsp27, or a biologically-active fragment or variant thereof. In a specific embodiment, the Hsp27 protein is capable of being in phosphorylated and non-phosphorylated forms. The polynucleotide can be an expression construct that provides for expression of an Hsp27 in a target cell. In one embodiment, the Hsp27 is a human Hsp27. In a specific embodiment, the human Hsp27 protein comprises the amino acid sequence of SEQ ID NO:1, or a biologically-active fragment or variant thereof. In a more specific embodiment, the human Hsp27 protein is encoded by a polynucleotide comprising the nucleotide sequence of SEQ ID NO:2. Compositions of the invention can also comprise other drugs or therapeutics that include, but are not limited to, donepezil (Aricept), galantamine (Razadyne), rivastigmine (Exelon), memantine (Akatinol), paclitaxel (Taxol), a statin, lithium, inhibitors of cyclin-dependent kinase 5, methylthionine chloride, and L-dopa.

The polypeptides of the present invention can be formulated into pharmaceutically-acceptable salt forms. Pharmaceutically-acceptable salt forms include the acid addition salts and include hydrochloric, hydrobromic, nitric, phosphoric, carbonic, sulphuric, and organic acids like acetic, propionic, benzoic, succinic, fumaric, mandelic, oxalic, citric, tartaric, maleic, and the like. Pharmaceutically-acceptable base addition salts include sodium, potassium, calcium, ammonium, and magnesium salts. Pharmaceutically-acceptable salts of the polypeptides of the invention can be prepared using conventional techniques.

The subject invention also concerns polynucleotide expression constructs that comprise a polynucleotide of the present invention comprising a nucleotide sequence encoding an Hsp27 polypeptide of the present invention. In one embodiment, the Hsp27 is a human Hsp27. In one embodiment, the polynucleotide encodes a polypeptide comprising the amino acid sequence shown in SEQ ID NO:1, or a fragment or variant thereof that exhibits Hsp27 biological activity. In a more specific embodiment, the human Hsp27 protein is encoded by a polynucleotide comprising the nucleotide sequence of SEQ ID NO:2. In a specific embodiment, the Hsp27 protein is capable of being in phosphorylated and non-phosphorylated forms.

As used herein, the term “expression construct” refers to a combination of nucleic acid sequences that provides for transcription of an operably linked nucleic acid sequence. As used herein, the term “operably linked” refers to a juxtaposition of the components described wherein the components are in a relationship that permits them to function in their intended manner. In general, operably linked components are in contiguous relation.

Expression constructs of the invention will also generally include regulatory elements that are functional in the intended host cell in which the expression construct is to be expressed. Thus, a person of ordinary skill in the art can select regulatory elements for use in, for example, bacterial host cells, yeast host cells, plant host cells, insect host cells, mammalian host cells, and human host cells. Regulatory elements include promoters, transcription termination sequences, translation termination sequences, enhancers, and polyadenylation elements.

An expression construct of the invention can comprise a promoter sequence operably linked to a polynucleotide sequence encoding a polypeptide of the invention. Promoters can be incorporated into a polynucleotide using standard techniques known in the art. Multiple copies of promoters or multiple promoters can be used in an expression construct of the invention. In a preferred embodiment, a promoter can be positioned about the same distance from the transcription start site as it is from the transcription start site in its natural genetic environment. Some variation in this distance is permitted without substantial decrease in promoter activity. A transcription start site is typically included in the expression construct.

For expression in animal cells, an expression construct of the invention can comprise suitable promoters that can drive transcription of the polynucleotide sequence. If the cells are mammalian cells, then promoters such as, for example, actin promoter, metallothionein promoter, NF-kappaB promoter, EGR promoter, SRE promoter, IL-2 promoter, NFAT promoter, osteocalcin promoter, SV40 early promoter and SV40 late promoter, Lck promoter, BMP5 promoter, TRP-1 promoter, murine mammary tumor virus long terminal repeat promoter, STAT promoter, or an immunoglobulin promoter can be used in the expression construct. In one embodiment, the expression construct comprises a promoter that can drive expression of a polynucleotide of the invention in neuronal or glial cells. Promoters effective in neurons that can be used in the present invention include gamma-aminobutyric acid type A receptor β₁ subunit promoter (U.S. Pat. No. 6,066,726), zebrafish HuC promoter (U.S. Published Application No. 20040093630), and cellular adhesion molecule ICAM-4 promoter (U.S. Pat. No. 5,753,502) (see also U.S. Pat. No. 6,040,172).

Expression constructs of the invention may optionally contain a transcription termination sequence, a translation termination sequence, signal polypeptide sequence, and/or enhancer elements. Transcription termination regions can typically be obtained from the 3′ untranslated region of a eukaryotic or viral gene sequence. Transcription termination sequences can be positioned downstream of a coding sequence to provide for efficient termination. Signal polypeptides are a group of short amino terminal sequences that encode information responsible for the relocation of an operably linked polypeptide to a wide range of post-translational cellular destinations, ranging from a specific organelle compartment to sites of protein action and the extracellular environment. Targeting a polypeptide to an intended cellular and/or extracellular destination through the use of operably linked signal polypeptide sequence is contemplated for use with the polypeptides of the invention. Chemical enhancers are cis-acting elements that increase gene transcription and can also be included in the expression construct. Chemical enhancer elements are known in the art, and include, but are not limited to, the CaMV 35S enhancer element, cytomegalovirus (CMV) early promoter enhancer element, and the SV40 enhancer element. DNA sequences which direct polyadenylation of the mRNA encoded by the structural gene can also be included in the expression construct.

Unique restriction enzyme sites can be included at the 5′ and 3′ ends of the expression construct to allow for insertion into a polynucleotide vector. As used herein, the term “vector” refers to any genetic element, including for example, plasmids, cosmids, chromosomes, phage, virus, and the like, which is capable of replication when associated with proper control elements and which can transfer polynucleotide sequences between cells. Vectors contain a nucleotide sequence that permits the vector to replicate in a selected host cell. A number of vectors are available for expression and/or cloning, and include, but are not limited to, pBR322, pUC series, M13 series, and pBLUESCRIPT vectors (Stratagene, La Jolla, Calif.). A specific vector contemplated by the present invention is an adeno-associated virus.

Polynucleotides, vectors, and expression constructs of the subject invention can be introduced into a cell by methods known in the art. Such methods include transfection, microinjection, electroporation, lipofection, cell fusion, calcium phosphate precipitation, and by biolistic methods. In one embodiment, a polynucleotide or expression construct of the invention can be introduced in vivo via a viral vector such as adeno-associated virus (AAV), herpes simplex virus (HSV), papillomavirus, adenovirus, and Epstein-Barr virus (EBV). Attenuated or defective forms of viral vectors that can be used with the subject invention are known in the art. Typically, defective virus is not capable of infection after the virus is introduced into a cell. Polynucleotides, vectors, and expression constructs of the invention can also be introduced in vivo via lipofection (DNA transfection via liposomes prepared from synthetic cationic lipids) (Felgner et al., 1987). Synthetic cationic lipids (LIPOFECTIN, Invitrogen Corp., La Jolla, Calif.) can be used to prepare liposomes to encapsulate a polynucleotide, vector, or expression construct of the invention. A polynucleotide, vector, or expression construct of the invention can also be introduced in vivo as naked DNA using methods known in the art, such as transfection, microinjection, electroporation, calcium phosphate precipitation, and by biolistic methods.

Polynucleotides and polypeptides of the subject invention can also be defined in terms of more particular identity and/or similarity ranges with those sequences exemplified herein. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence exemplified herein. Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

The subject invention also contemplates those polynucleotide molecules (encoding polypeptides of the invention) having sequences which are sufficiently homologous with the polynucleotide sequences encoding a polypeptide of the invention so as to permit hybridization with that sequence under standard stringent conditions and standard methods (Maniatis, T. et al., 1982). As used herein, “stringent” conditions for hybridization refers to conditions wherein hybridization is typically carried out overnight at 20-25 C below the melting temperature (Tm) of the DNA hybrid in 6×SSPE, 5×Denhardt's solution, 0.1% SDS, 0.1 mg/ml denatured DNA. The melting temperature is described by the following formula (Beltz, G. A. et al., 1983):

Tm=81.5 C+16.6 Log [Na+]+0.41 (% G+C)−0.61(% formamide)−600/length of duplex in base pairs.

Washes are typically carried out as follows:

(1) Twice at room temperature for 15 minutes in 1×SSPE, 0.1% SDS (low stringency wash).

(2) Once at Tm-20 C for 15 minutes in 0.2×SSPE, 0.1% SDS (moderate stringency wash).

As used herein, the terms “nucleic acid” and “polynucleotide sequence” refer to a deoxyribonucleotide or ribonucleotide polymer in either single- or double-stranded form, and unless otherwise limited, would encompass known analogs of natural nucleotides that can function in a similar manner as naturally-occurring nucleotides. The polynucleotide sequences include both the DNA strand sequence that is transcribed into RNA and the RNA sequence that is translated into protein. The polynucleotide sequences include both full-length sequences as well as shorter sequences derived from the full-length sequences. It is understood that a particular polynucleotide sequence includes the degenerate codons of the native sequence or sequences which may be introduced to provide codon preference in a specific host cell. The polynucleotide sequences falling within the scope of the subject invention further include sequences which specifically hybridize with the sequences coding for a polypeptide of the invention. The polynucleotide includes both the sense and antisense strands as either individual strands or in the duplex.

In one embodiment, an effective amount of a polypeptide, polynucleotide, and/or composition of the present invention is administered to a patient having a neurodegenerative disorder and who is in need of treatment thereof. The patient can be a human or other mammal, such as a dog, cat, or horse, or other animals having the disorder. Means for administering and formulating polypeptides and polynucleotides for administration to a patient are known in the art, examples of which are described herein. Polypeptides, polynucleotides, and/or compositions of the invention can be delivered to a cell either through direct contact of polypeptide or polynucleotide with the cell or via a carrier means. In one embodiment, a polypeptide or polynucleotide of the invention comprises an attached group that enhances cellular uptake of the polypeptide. In one embodiment, the polypeptide or polynucleotide is attached to an antibody that binds to a targeted cell. In another embodiment, the polypeptide or polynucleotide is encapsulated in a liposome. Polypeptides can also be delivered using a polynucleotide that encodes a subject polypeptide. Any polynucleotide having a nucleotide sequence that encodes a polypeptide of the invention is contemplated within the scope of the invention. In one embodiment, the polynucleotide is delivered to the cell where it is taken up and the polynucleotide is transcribed into RNA and the RNA is translated into the encoded polypeptide.

Therapeutic application of the subject polypeptides and polynucleotides, and compositions containing them, can be accomplished by any suitable therapeutic method and technique presently or prospectively known to those skilled in the art. The polypeptides and polynucleotides can be administered by any suitable route known in the art including, for example, oral, intramuscular, intraspinal, intracranial, nasal, rectal, parenteral, subcutaneous, or intravenous routes of administration. Administration of the polypeptides and polynucleotides of the invention can be continuous or at distinct intervals as can be readily determined by a person skilled in the art.

Compounds and compositions useful in the subject invention can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention will be formulated such that an effective amount of the bioactive polypeptide or polynucleotide is combined with a suitable carrier in order to facilitate effective administration of the composition. The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically acceptable carriers and diluents which are known to those skilled in the art. Examples of carriers or diluents for use with the subject polypeptides and polynucleotides include, but are not limited to, water, saline, oils including mineral oil, ethanol, dimethyl sulfoxide, gelatin, cyclodextrans, magnesium stearate, dextrose, cellulose, sugars, calcium carbonate, glycerol, alumina, starch, and equivalent carriers and diluents, or mixtures of any of these. Formulations of the polypeptide or polynucleotide of the invention can also comprise suspension agents, protectants, lubricants, buffers, preservatives, and stabilizers. To provide for the administration of such dosages for the desired therapeutic treatment, pharmaceutical compositions of the invention will advantageously comprise between about 0.1% and 45%, and especially, 1 and 15% by weight of the total of one or more of the polypeptide or polynucleotide based on the weight of the total composition including carrier or diluent.

The compounds and molecules of the subject invention can also be administered utilizing liposome technology, slow release capsules, implantable pumps, and biodegradable containers. These delivery methods can, advantageously, provide a uniform dosage over an extended period of time.

The subject polypeptides and polynucleotides of the invention can also be modified by the addition of chemical groups, such as PEG (polyethylene glycol). PEGylated polypeptides typically generate less of an immunogenic response and exhibit extended half-lives in vivo in comparison to polypeptides that are not PEGylated when administered in vivo. Methods for PEGylating proteins and polypeptides known in the art (see, for example, U.S. Pat. No. 4,179,337). The subject polypeptides and polynucleotides can also be modified to improve cell membrane permeability. In one embodiment, cell membrane permeability can be improved by attaching a lipophilic moiety, such as a steroid, to the polypeptide or polynucleotide. Other groups known in the art can be linked to polypeptides or polynucleotides of the present invention.

The subject invention also concerns a packaged dosage formulation comprising in one or more packages, packets, or containers at least one polypeptide or polynucleotide and/or composition of the subject invention formulated in a pharmaceutically acceptable dosage. The package can contain discrete quantities of the dosage formulation, such as tablet, capsules, lozenge, and powders. The quantity of polypeptide or polynucleotide in a dosage formulation and that can be administered to a patient can vary from about 1 mg to about 5000 mg, or about 1 mg to about 2000 mg, or more typically about 1 mg to about 500 mg, or about 5 mg to about 250 mg, or about 10 mg to about 100 mg.

The subject invention also concerns kits comprising in one or more containers a composition, compound, or molecule of the present invention. In one embodiment, a kit contains a polypeptide or polynucleotide of the present invention. In a specific embodiment, a kit comprises a polypeptide comprising the amino acid sequence shown in SEQ ID NO:1, or a fragment or variant of the polypeptide that exhibits Hsp27 activity. In a specific embodiment, the Hsp27 protein is capable of being in phosphorylated and non-phosphorylated forms. In another embodiment, a kit comprises a polynucleotide comprising a nucleotide sequence shown in SEQ ID NO:2. A kit of the invention can also comprise one or more compounds, biological molecules, or drugs. In one embodiment, a kit of the invention can comprise a polypeptide or polynucleotide of the invention, and optionally comprises one or more of a drug or composition used in treating a neurodegenerative disease or condition. Examples of other drugs or therapeutics include, but are not limited to, donepezil (Aricept), galantamine (Razadyne), rivastigmine (Exelon), memantine (Akatinol), paclitaxel (Taxol), a statin, lithium, inhibitors of cyclin-dependent kinase 5, methylthionine chloride, and L-dopa.

Polypeptides having substitution of amino acids other than those specifically exemplified in the subject polypeptides are also contemplated within the scope of the present invention. For example, non-natural amino acids can be substituted for the amino acids of a polypeptide of the invention, so long as the polypeptide having substituted amino acids retains substantially the same activity as the polypeptide in which amino acids have not been substituted. Examples of non-natural amino acids include, but are not limited to, ornithine, citrulline, hydroxyproline, homoserine, phenylglycine, taurine, iodotyrosine, 2,4-diaminobutyric acid, a-amino isobutyric acid, 4-aminobutyric acid, 2-amino butyric acid, γ-amino butyric acid, e-amino hexanoic acid, 6-amino hexanoic acid, 2-amino isobutyric acid, 3-amino propionic acid, norleucine, norvaline, sarcosine, homocitrulline, cysteic acid, t-butylglycine, t-butylalanine, phenylglycine, cyclohexylalanine, β-alanine, fluoro-amino acids, designer amino acids such as β-methyl amino acids, C-methyl amino acids, N-methyl amino acids, and amino acid analogues in general. Non-natural amino acids also include amino acids having derivatized side groups. Furthermore, any of the amino acids in the protein can be of the D (dextrorotary) form or L (levorotary) form.

Amino acids can be generally categorized in the following classes: non-polar, uncharged polar, basic, and acidic. Conservative substitutions whereby a polypeptide having an amino acid of one class is replaced with another amino acid of the same class fall within the scope of the subject invention so long as the polypeptide having the substitution still retains substantially the same biological activity as a polypeptide that does not have the substitution. Table 1 below provides a listing of examples of amino acids belonging to each class.

TABLE 1

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

Single letter amino acid abbreviations are defined in Table 2.

TABLE 2

| Letter Symbol | Amino Acid |
|---|---|
| A | Alanine |
| B | Asparagine or aspartic acid |
| C | Cysteine |
| D | Aspartic Acid |
| E | Glutamic Acid |
| F | Phenylalanine |
| G | Glycine |
| H | Histidine |
| I | Isoleucine |
| K | Lysine |
| L | Leucine |
| M | Methionine |
| N | Asparagine |
| P | Proline |
| Q | Glutamine |
| R | Arginine |
| S | Serine |
| T | Threonine |
| V | Valine |
| W | Tryptophan |
| Y | Tyrosine |
| Z | Glutamine or glutamic acid |

The methods and compositions of the present invention can be used in the treatment of humans and other animals. The other animals contemplated within the scope of the invention include domesticated, agricultural, or zoo- or circus-maintained animals. Domesticated animals include, for example, dogs, cats, rabbits, ferrets, guinea pigs, hamsters, pigs, monkeys or other primates, and gerbils. Agricultural animals include, for example, horses, mules, donkeys, burros, cattle, cows, pigs, sheep, and alligators. Zoo- or circus-maintained animals include, for example, lions, tigers, bears, camels, giraffes, hippopotamuses, and rhinoceroses.

### Materials and Methods

Antibodies:

Anti-Hsp27 and hTau-150 rabbit polyclonal antibodies were purchased from Santa Cruz Biotechnology, Inc. Rabbit polyclonal anti-human tau was purchased from Dako USA. Rabbit polyclonal anti-pS199/S202 tau was purchased from Anaspec, Inc. NeuN antibody was purchased from Millipore, Co. Mouse monoclonal anti-His antibody was purchased from Invitrogen Co. Secondary antibodies conjugated to HRP, and anti-goat secondary antibody conjugated to a fluorophore (594 nm) were purchased from Southern Biotech. or AlexaFluor (Molecular Probes) were optimized and diluted accordingly as described below.

Virus:

Adeno-associated virus serotype 9 (AAV9)-expressing wtHsp27 was a gift from Dr. Todd Golde. AAV9-EGFP was also a gift from Dr. Kevin Nash. AAV9-3×S/D Hsp27 was a gift from Dr. Grant Mauk.

Recombinant Protein:

Production and purification of recombinant proteins was based on protocols previously described (Berrow et al. 2006). pET28 vectors carrying His-tagged gene sequences of tau, wtHsp27, and 3×S/D Hsp27 were transformed into the Escherichia coli strain BL21 (DE3) Codon Plus. The latter sequence, 3×S/D Hsp27, consists of the wtHsp27 sequence with amino acid substitutions: S15D, S78D, and S82D. LB medium containing kanamycin was inoculated with a respective stationary overnight culture. Cultures were grown at 30° C. to an OD₆₀₀ of 0.5. Protein expression was induced by addition of 1 mM isopropyl-β-D-thiogalactopyranoside (IPTG) for 4 hours. Cells were harvested by centrifugation at 4000 g for 15 minutes at 4° C. Pelleted cells were lysed by sonication. All proteins were purified via a succession of Ni-NTA-sepharose chromatography (equilibrated in 50 mM NaH₂PO₄, 500 mM NaCl, 30 mM imidazole (pH 7.5), elution by a step gradient to 300 mM imidazole). The purity of all proteins was verified on a Coomassie-Brilliant-Blue stained SDS-polyacrylamide gel.

Sucrose Cushion (SC):

Sucrose-PBS solutions of increasing densities (20-50%) were layered in ultracentrifuge tubes. Recombinant proteins at a concentration of 1.2 μg were incubated in 20 mM HEPES or sterile PBS for 30 min at RT. Samples were then added to the top of the sucrose layers and centrifuged at 100,000 g.

Western Blot (WB):

Western blot experiments were performed as previously described (Dickey et al. 2009). Samples were mixed with either a 6×WB sample buffer (50 mM Tris, 10% SDS for SC samples) or a 2× Laemmli sample buffer (Bio-Rad; for IP samples), boiled for 10 minutes, and loaded onto 10% Tris-Glycine gels (Invitrogen) or 18-well, 10% Criterion gels (Bio-Rad). Gel proteins were transferred onto nitrocellulose membranes, which were blocked for 60 min at RT in 7% milk. Then, membranes were incubated with primary antibodies at a dilution recommended by the manufacturer.

Immunoprecipitation (IP):

IP experiments were performed as described in (Dickey et al. 2009). Purified recombinant tau proteins were incubated and agitated with BSA (as a control), wtHsp27, or 3×S/D Hsp27 in Protein Base Buffer (PBB: 57.5 mM Na₂HPO₄, 334.42 mM NaCl, pH 8.0) for 30 min at room temperature (RT) and co-immunoprecipitated with anti-Hsp27 antibody for 1 hour at RT. Protein A slurry (50 μL) was added and the mixture was incubated at RT for 2 hours. Samples were centrifuged and Protein A beads precipitated. Pellets were washed four times with PBB buffer. The precipitates were subjected to western blot detection of Hsp27 and tau using His antibody or tau antibody (DAKO).

Animals:

All procedures involving experimentation on animal subjects were done in accord with the guidelines set forth by the University of South Florida's Institutional Animal Care and Use Committee (IACUC). The rTg4510 and parental mice were maintained and genotyped as previously described (Santacruz et al. 2005). For immunohistochemistry experiments, mice were injected with AAV9-expressing vectors at 4 months, and their brains were harvested two months post-injection. Alternatively, mice were injected with AAV9-expressing vectors at 1.5 months, and their brains were harvested 2.5 months post-injection for electrophysiology experiments. The surgical procedure was performed as previously described (Carty et al. 2008) with some modifications. Twenty-five percent by mass Mannitol (Fisher) was delivered to mice by intra-peritoneal injections 15 minutes pre-surgery. Using isofluorane, mice were anesthetized, and the cranium was exposed with a small incision along the skin covering the medial sagittal plane. Holes were drilled through the cranium over the coordinates to reach the injection sites measured with a stereotaxic apparatus from Bregma at: −1.5 mm anteroposterior, 2/−2 mm bilateral, and 3.0 mm vertical for frontal cortex; 2.2 mm anteroposterior, 2.2/−2.2 mm bilateral, and 2.3 vertical for CA1; and 1.0 mm anteroposterior, 1.0/−1.0 mm bilateral, and 2.5 mm vertical for the lateral ventricles. Burr holes were drilled using a dental drill bit (SSW HP-3; SSWhiteBurs) or a 21-gauge needle (BD). A 2 μl total volume of each of the viral vectors in sterile PBS at a concentration of >10¹¹ vg/ml were dispensed into the each injection site at a rate of 5 μl/min using convection-enhanced delivery (CED) (Bobo et al. 1994). The needle was removed after two min of the injection. The incision was then cleaned and closed with Vetbond solution (3M). The animals recovered within 10 min and were housed singly until the time their brains were harvested. CED of AAV9 was performed with a modified 26-gauge blunt needle (Hamilton) attached to a 10 μl syringe (Hamilton). Needles were modified by attaching standard polyimide-coated, flexible-fused silica capillary tubing, (100±04 μm; TSP100170; Polymicro Technologies) into the needle shaft with Super Glue (Loctite), allowing 1 mm to be exposed outside the needle and 3 mm to remain inside.

Immunohistochemistry:

Mouse brains were harvested for immunohistochemical analysis as previously described (Dickey et al. 2009). Briefly, mice were overdosed with pentobarbital and perfused transcardially with 0.9% normal saline solution. Brains were removed and immersed in 4% para-formaldehyde. Fixed brains were cryo-protected in successive 24-hour increments of 10%, 20%, and 30% sucrose gradients as previously described (Gordon et al. 2002). Brains were frozen on a temperature-controlled freezing stage, coronally sectioned (25-50 μm) on a sliding microtome, and stored in a solution of PBS containing 0.02% NaN₃ at 4° C. Immunostaining was performed following floating section procedure previously described (Gordon et al. 1997) with minor modifications. Immunostaining procedure began by immersing brain sections in a 3% solution of H₂O₂ for 15 min at RT to block endogenous peroxidases. Sections were washed in PBS and permeabilized in blocking buffer (4% donkey serum, 1.83% Lysine, 2% Triton X-100) for 30 min at RT and then incubated overnight at 4° C. with anti-Hsp27 antibody at a 1:2000 dilution. After washing with PBS, sections were incubated with biotinylated anti-goat secondary antibody for two hours and then with streptavidin-peroxidase. The peroxidase reactions consisted of 1.4 mM diaminobenzidine with 0.03% hydrogen peroxide in PBS for 5 min. Brain sections for immunofluorescence were blocked as described above and incubated overnight at 4° C. with biotinylated-NeuN (1:100), anti-pS199/S202 tau (1:1000), and anti-Hsp27 (1:1000) primary antibodies. Slides were washed and then incubated with AlexaFluor secondary antibodies for two hours at RT; dilutions were determined according to the manufacturer. Finally, stained sections were mounted on glass slides and imaged. Non-specific reaction product formation was negligible as assessed by omitting the primary antibody. NeuN staining was selected as a neuronal marker. Due to the potential loss of NeuN recognition in damaged neurons (McPhail et al. 2004; Unal-Cevik et al. 2004; Collombet et al. 2006; Kienzler et al. 2009), neuronal selection may have excluded damaged cells.

Microscopy:

Immunohistochemically-stained sections were brightfield imaged using the Zeiss Axiolmager.Z1 and AxioVision software with the 5×/0.16 dry ECPlanApo objective. Images covering the entire brain sections were captured using an AxioCam MRc5 camera, and stitched together as a single image using the Zeiss Panorama program. Immunofluorescently-stained brain sections were imaged using the Leica TCS SP2 laser scanning confocal microscope, which is inverted and has a motorized z-stage (z-Galvo and z-Wide). The fields examined were selected based on the following criteria: regions in the hippocampus that were Hsp27- or GFP-positive. Z-stacked images of the selected fields were captured using a 40×/1.25-0.75 PLAN APO Oil objective and a Leica photomultiplier tube. Lasers used were 405 diode (for NeuN-positive signal represented in blue), Argon (for Hsp27/GFP-positive signal in green), and Red HeNe (for tau-positive signal in red).

Image Analysis:

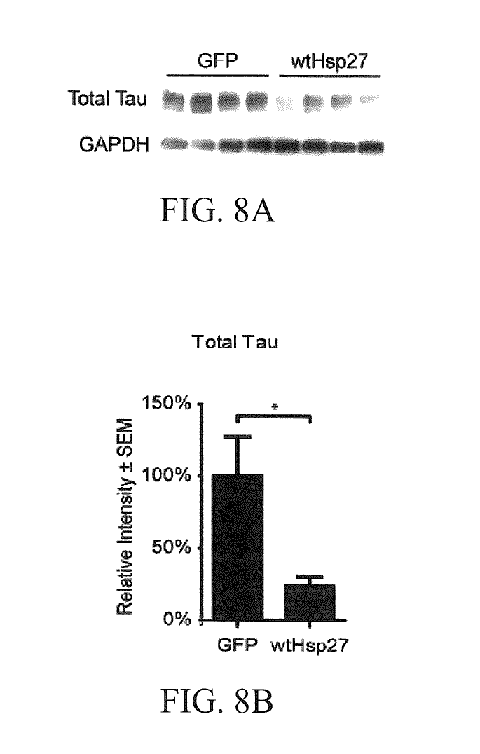

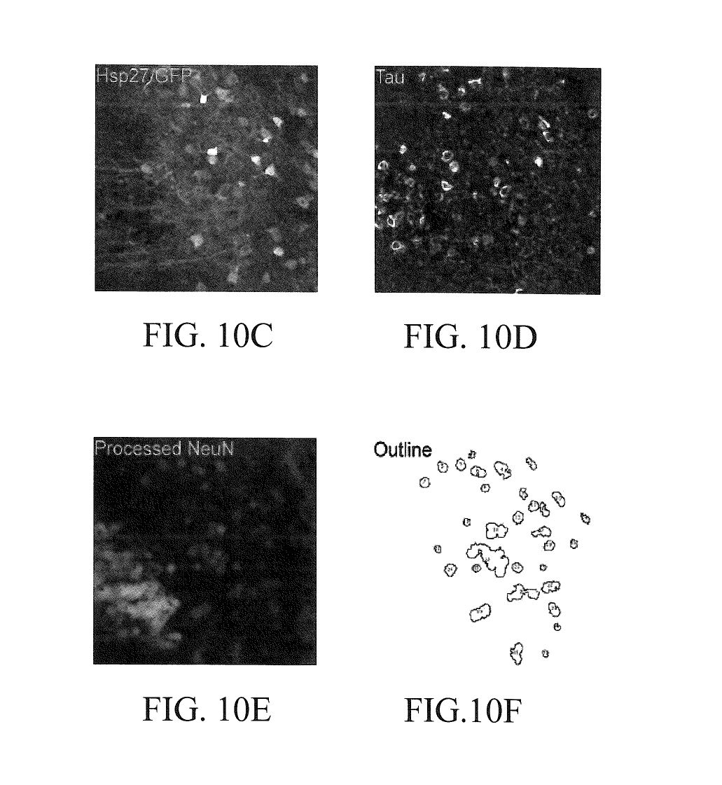

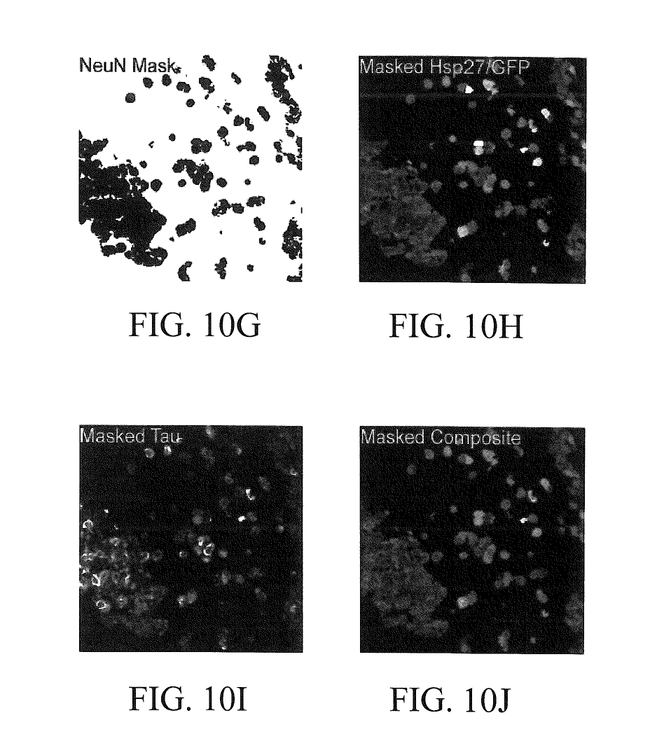

Tau signal quantification was performed using ImageJ (Bolte and Cordelieres 2006; Rasband, 2009). The signals from each channel from z-stacked images for the ROI were summed into a final image that was imported from the Leica software into ImageJ (FIG. 8A). The three channels were split into independent images for NeuN, Hsp27 or GFP, and tau (FIGS. 10B-10D). To obtain a mask that revealed only NeuN-positive regions, the blue channel was processed by firstly removing outliers resulting in an image highlighting the NeuN-positive regions as shown in FIG. 10E (Process>Noise>Remove Outliers>radius 10 pixels, threshold of 5, and remove Bright). The threshold for this image was set to a grayscale range of 70-255, the outline of which is presented in FIG. 10F. We selected the Area and the Integrated Density (Int. Den.—Analyze>Set Measurements) and redirected the calculations to the red and green channels. The following adjustments were then made in Analyze>Analyze Particles: we further specified our NeuN-specific ROI to areas greater that 100 pixels² and circularity of 0.00-1.00, which increased the selectivity of our measurements to neurons. We also excluded areas along the edges of the field. Since the neuron somas were sometimes dissected by the z-stack, we included the area within the blue delineations, which correspond to areas where neuron bodies are present. The Int. Den. values for each NeuN region defined in the outline were divided by the Area of the same regions, which resulted in the pixel density per area for tau and Hsp27/GFP. We defined the signal above background as those values that were two standard deviations above the mean. We then determined the mean of the tau signals above background in NeuN-positive regions for each condition: GFP or Hsp27-expressing brains.

Electrophysiology Experiments:

Mice were decapitated and the brains were rapidly removed and briefly submerged in ice-cold cutting saline (110 mm sucrose, 60 mm NaCl, 3 mm KCl, 1.25 mm NaH₂PO₄, 28 mm NaHCO₃, 0.5 mm CaCl₂, 5 mm d-glucose, and 0.6 mm ascorbate). All solutions were saturated with 95% O₂ and 5% CO₂. Whole brains were then dissected on cutting solution-soaked filter paper and mounted on a glass platform resting on ice. Hippocampal slices (400 μm) were prepared on a vibratome and allowed to equilibrate in a 50% cutting saline and 50% artificial cerebrospinal fluid solution (ACSF; 125 mm NaCl, 2.5 mm KCl, 1.24 mm NaH₂PO₄, 25 mm NaHCO₃, 10 mm d-glucose, 2 mm CaCl₂, and 1 mm MgCl₂) at room temperature for a minimum of 10 min. Slices were transferred to an interface chamber supported by a nylon mesh and allowed to recover for a minimum of 1.5 hrs prior to recording. Slices were perfused in ACSF at one ml/min. Field excitatory post-synaptic potentials (fEPSPs) were obtained from area CA1 stratum radiatum. Stimulation was supplied with a bipolar Teflon-coated platinum electrode and a recording was obtained with the use of a glass microelectrode filled with ACSF (resistance 1-4 mO). fEPSPs were generated using a 0.1 msec biphasic pulse delivered every 20 sec. After a consistent response to a voltage stimulus was established for a 5-10 min period, threshold voltage for evoking a fEPSP was determined and the voltage was then increased incrementally every 0.5 mV until the maximum amplitude of the fEPSP was reached. These data were used to create the I/O curve. A fEPSP baseline response, defined as 50% of the stimulus voltage used to produce the maximum fEPSP amplitude as determined by the I/O curve, was then recorded for 20 min. The tetanus used to evoke CA1 LTP consisted of a theta-burst stimulation (tbs) protocol. The tbs consisted of five trains of four pulse bursts at 200 Hz separated by 200 ms, repeated six times with an inter-train interval of 10 s. Following tbs, fEPSPs evoked by baseline stimulus were recorded for 60 min. Potentiation was measured as the normalized increase of the mean fEPSP descending slope following TBS normalized to the mean fEPSP descending slope for the duration of the baseline recording. Experimental results were obtained from those slices that exhibited stable baseline synaptic transmission for a minimum of 20 min prior to the delivery of the LTP-inducing stimulus. Student T-tests were performed during the first and last 5 min of the recordings.

Dynamic Light Scattering:

To analyze the effects of Hsp27 on in vitro fibril formation by tau we used correlated DLS (Hill et al. 2009). Recombinant tau (250 mM) was suspended in buffer (20 mM Tris-HCl, 100 mM NaCl, pH=7.4), with Hsp27 at a stoichiometric ratio of 50 parts tau to 1 part Hsp27. All proteins were filtered through 0.2 mm syringe filters (Anotop) and passed through 100 kD molecular cut-off filters (Nanosep). Heparin, at ¼ the molar concentration of tau, was added to initiate aggregation (Barghorn et al. 2004). Samples were placed in glass cuvettes and aggregation kinetics at 37° C. were monitored in 3 min intervals using DLS (Malvern Zetasizer Nano S). The low molar ratios of Hsp27 to tau (typ. 1:50) made Hsp27 contributions to the DLS signal negligible.

Atomic Force Microscopy:

Samples were diluted in 10× buffer, and 75 uL of the solution were deposited onto freshly cleaved mica for 15 minutes, rinsed with deionized water, and dried with dry nitrogen. Samples were imaged in air with an MFP-3D atomic-force microscope (Asylum Research, Santa Barbara, Calif.) using PPP-FMR (Nanosensor, Neuchatel, Switzerland) silicon tips with nominal tip radii 7 nm. The cantilever was driven at 60-70 kHz in alternating current mode and a scan rate of 0.5 Hz, acquiring images at a 1024×1024-pixel resolution.

Statistical Analyses:

Statistical analyses were performed by Student T-tests as indicated in the figure legends.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1

### Wild-Type and Mock-Phosphorylated Hsp27 Bind to and Abrogate the Aggregation of Tau

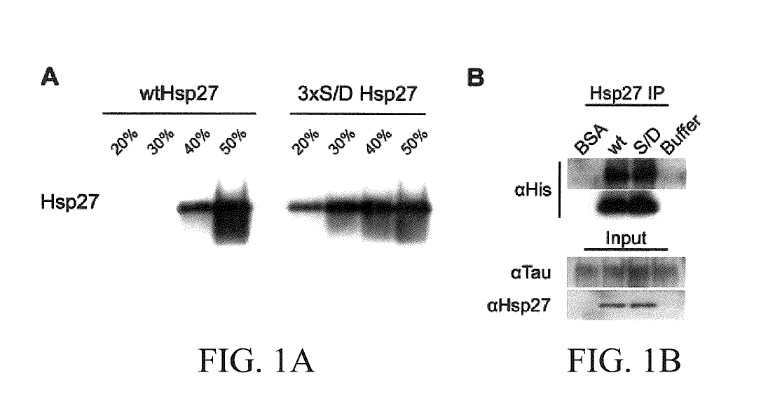

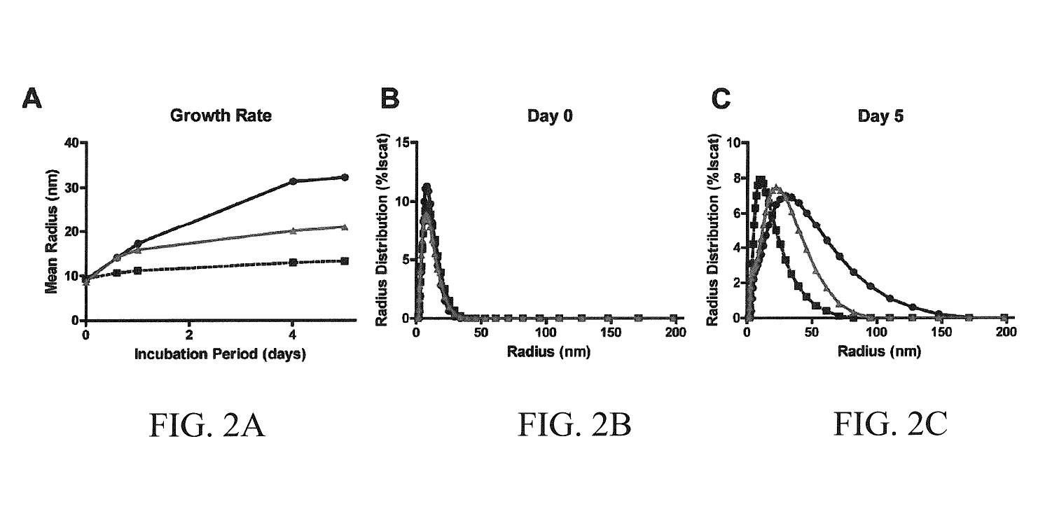

We generated two recombinant variants of Hsp27: wild-type Hsp27 (wtHsp27) and a mutant form of Hsp27 where serines 15, 78, and 82 are substituted by aspartates (3×S/D Hsp27). We analyzed the oligomerization properties of these Hsp27 variants with 20%-50% sucrose cushioning. Sucrose fractions were collected after ultracentrifugation and Hsp27 was detected by western blot (FIG. 1A). Wild-type Hsp27 was predominantly found in the 50% fraction, whereas 3×S/D Hsp27 was distributed throughout all sucrose fractions. This confirmed that phosphorylation of Hsp27 abrogates multimer formation, whereas wild-type Hsp27 is more prone to oligomerization (Lelj-Garolla and Mauk). Both Hsp27 variants were able to bind tau, as demonstrated by co-immunoprecipitation (FIG. 1B).

We then tested whether tau aggregation could be altered by both Hsp27 variants using atomic force microscopy (AFM) and dynamic light scattering (DLS) (Reynolds et al. 2005). DLS showed reductions in the growth rate of tau filament formation over a 5 day period (FIG. 2A). No rapid or spontaneous aggregation of tau was observed at day 0 (FIG. 2B). However, after incubation for 5 days, both wt- and 3×S/D Hsp27 reduced tau aggregation at sub-stoichiometric ratios (35:1) compared to tau incubated with buffer alone (FIG. 2C). However, wildtype Hsp27 (r~ 10 nm; day 5) was more effective than 3×S/D Hsp27 (r~ 20 nm; day 5) at abrogating tau aggregation (FIG. 2A). These samples were then incubated an additional 10 days, and AFM was used to image tau structures with or without each Hsp27 variant (FIG. 2D). Tau incubated with heparin alone formed robust filamentous aggregates. Conversely, tau incubated with wildtype Hsp27 or 3×S/D Hsp27 failed to form filaments, but intermediate aggregates of tau were observed in the presence of both variants. Consistent with our DLS findings, wildtype Hsp27 more potently reduced tau aggregation compared to 3×S/D Hsp27 (FIG. 2D).

### Example 2

### Convection Enhanced Delivery of AAV9 Facilitates Robust Distribution of Gene Product in the Hippocampus

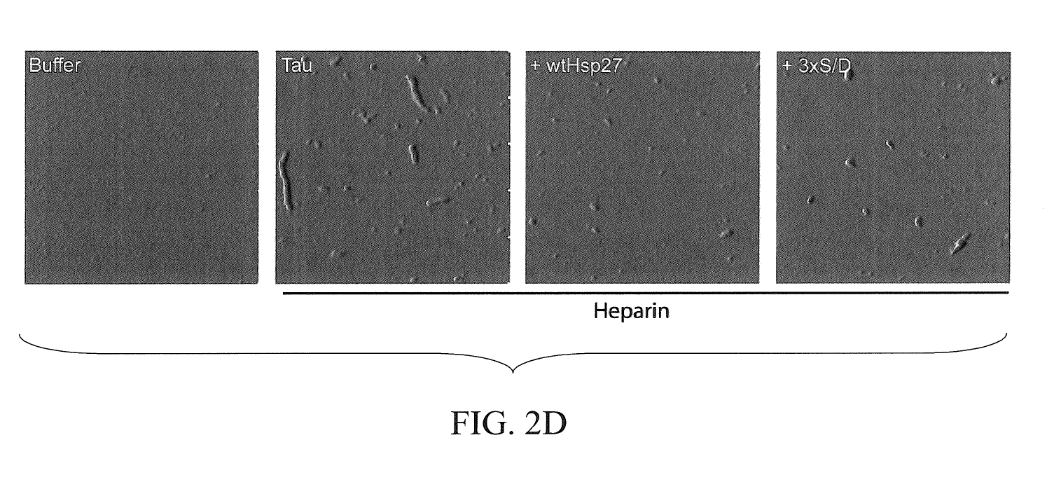

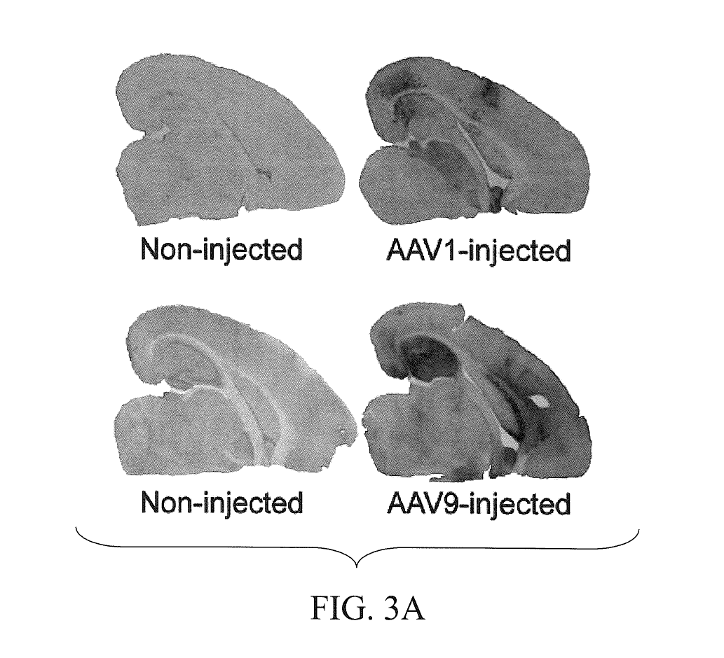

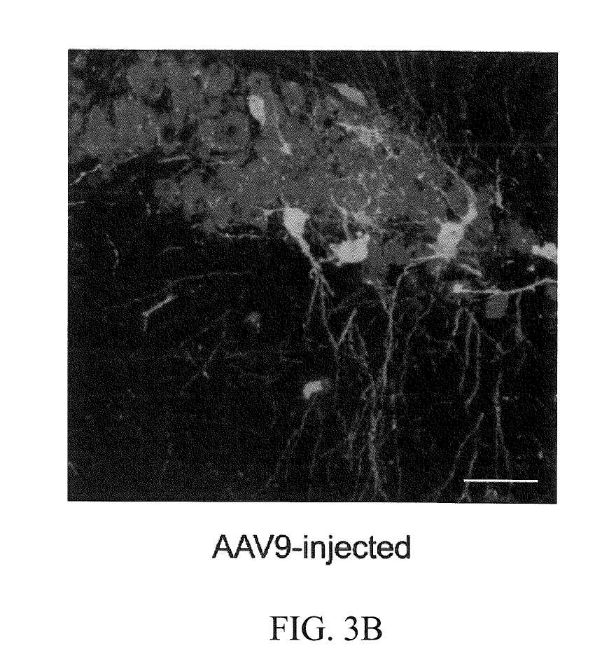

Since both Hsp27 variants were capable of modifying tau biology in vitro, we sought to determine their impact in the brain. We implemented an adeno-associated viral (AAV) expression system with the goal of delivering both Hsp27 variants to the rTg4510 transgenic mouse model of tauopathy (Santacruz et al. 2005). To test distribution and efficacy of this approach, AAV1 and AAV9 particles expressing wtHsp27 and 3×S/D Hsp27 were generated and delivered to the brain using two different approaches based on previous studies: AAV1 was somatically delivered into the ventricular space of non-transgenic P0 pups as previously described (Levites et al. 2006); AAV9 was delivered into the CA1 region of the hippocampi and frontal cortices of adult, non-transgenic mice using convection enhanced delivery (CED) combined with intraperitoneal injections of mannitol prior to surgery (Fu et al. 2003; Hadaczek et al. 2006; Cearley et al. 2008). Eight weeks after injections, mice were killed and distribution of Hsp27 was assessed by immuno-histochemistry. Hippocampal injection of AAV9 caused robust distribution throughout the region, but the cortical injection was much less consistent across animals was quite variable (FIG. 3A). While the AAV1 delivery did result in greater overall spread of the viral particles, the intensity of staining in any one region was unremarkable. Moreover, hippocampal staining was minimal (FIG. 3A). Although distribution with AAV9 in the hippocampus was robust and primarily neuronal, immuno-fluorescent staining showed that all neurons (NeuN; blue) were not transduced by virus (Hsp27; green; FIG. 3B). These data suggested that AAV9 administration to the hippocampus using CED and mannitol would provide the most consistent and robust signal in the hippocampus, but would require analyses of individual cells to assess changes in tau levels.

### Example 3

### Tau Clearance by Hsp27 is Dependent on Phosphorylation Dynamics

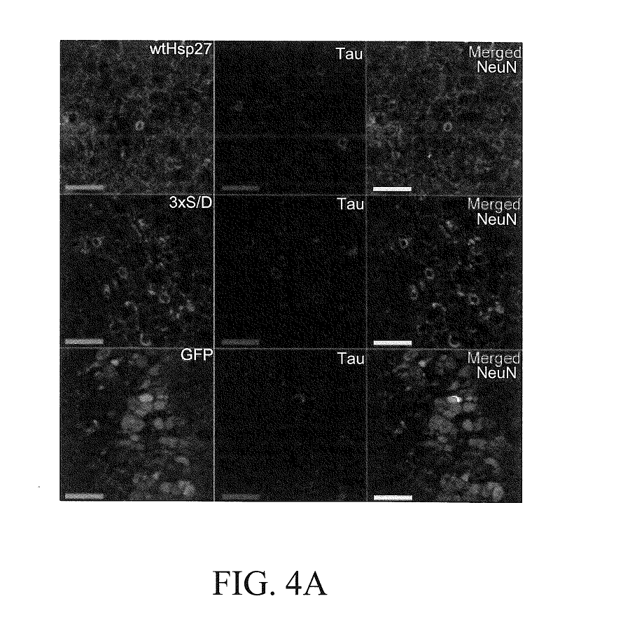

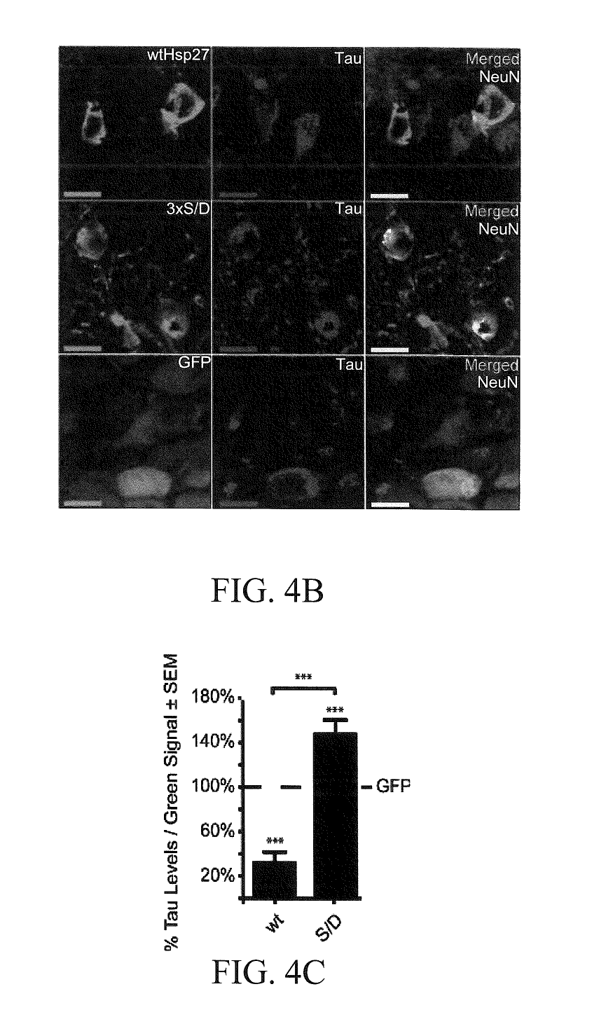

We used the rTg4510 transgenic mouse model to test the effects of both Hsp27 variants on tau pathology in neurons by bilaterally injecting their hippocampi with AAV9-expressing wtHsp27, 3×S/D Hsp27, or GFP (FIGS. 3A-3B). Since these mice begin to develop tangle pathology as early as 3 months of age (Dickey et al. 2009), and we already demonstrated that all neurons were not transduced by AAV, we initiated these studies in 4 month-old mice and harvested tissues two months later to ensure that most hippocampal neurons would have tau accumulation (Dickey et al. 2009). This design allowed us to assess the tau burden in neurons that were successfully transduced with viral particles encoding each Hsp27 variant or GFP by triple labeling tissue sections with antibodies specific for NeuN (blue), Hsp27 (green), and tau (red; FIGS. 4A-4B). Interestingly, we found very few wtHsp27-expressing neurons that also contained tau aggregates, suggesting that wtHsp27 facilitated tau clearance (FIG. 4B). This was in stark contrast to neurons positively stained for Hsp27 from mice receiving the 3×S/D Hsp27 variant, in these mice, tau levels were actually higher relative to mice injected with GFP-AAV. Quantification of tau levels in ˜300 transduced neurons from each group showed that tau was reduced by 66% in wtHsp27-expressing neurons compared to GFP-expressing neurons (FIG. 4D), whereas neurons expressing 3×S/D Hsp27 had 50% higher levels of tau compared to GFP-expressing neurons (FIG. 4C; ***p<0.001).

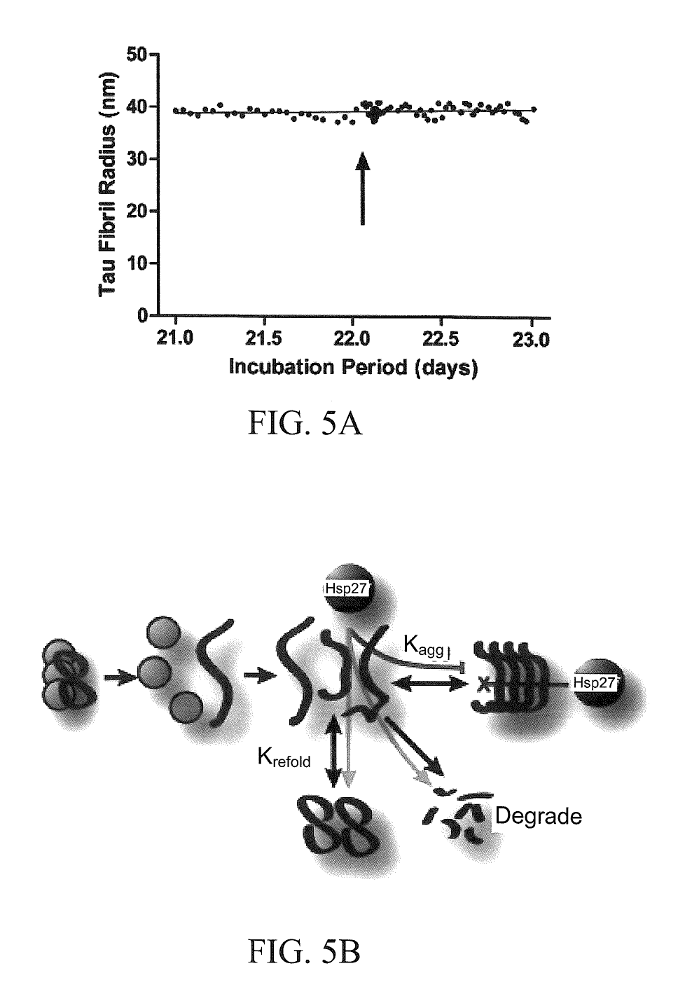

WtHsp27 was able to reduce tau levels in neurons; however, it was unclear whether it was preventing tau aggregation or actively disrupting pre-formed tau structures. DLS was used to specifically address this question. Recombinant tau was incubated with heparin for 22 days before the addition of recombinant wtHsp27 at a molar ratio equivalent to that used in FIGS. 2A-2D. Wild-type Hsp27 was unable to disrupt pre-formed aggregates (FIG. 5A), suggesting that in order for Hsp27 to impact neuronal function in the rTg4510 model it should be administered at a time prior to robust tau aggregation, which would be much earlier than 4 months of age. This result indicates that for Hsp27 to be effective at modulating tau aggregation, it must interact with tau prior to filament formation, at a point when tau is in an intermediate state (FIG. 5B). Thus, Hsp27 can block the tendency of tau to aggregate, facilitating its entry into degradation or refolding pathways. However, Hsp27 cannot disaggregate preformed tau fibrils.

Thus, a bias for healthy neurons was indeed introduced by using NeuN in our imaging studies, since damaged neurons may lose antigenicity for NeuN (McPhail et al. 2004; Unal-Cevik et al. 2004; Collombet et al. 2006; Kienzler et al. 2009). However, since damaged neurons are likely a result of tau aggregation, we would speculate that Hsp27 would not impact damaged neurons since it cannot disaggregate pre-formed tau filaments (FIG. 5A), making such a bias unlikely to affect the interpretation of our results.

### Example 4

### Hippocampal Plasticity Deficits in the rTg4510 Mice are Rescued by AAV9-Hsp27 Injections

The next goal of these studies was to determine the physiological outcome of Hsp27 over-expression in the brain. Since Hsp27 was unable to disaggregate pre-formed tau filaments, we speculated that any effects Hsp27 might have on the tau-induced phenotype of the rTg4510 mice would be most pronounced if administration of Hsp27 was initiated prior to any tangle formation. Previous work showed that tangle formation in the rTg4510 hippocampus begins at ˜2-3 months, and neuronal loss caused by this aberrant tau aggregation is only evident after 5 months. Thus, the CA1 subfields of hippocampi of 2-month old rTg4510 mice and wildtype littermates (NTG) were injected with saline or AAV9 encoding either wtHsp27, 3×S/D Hsp27, or GFP, with the goal being to test the effects of Hsp27 over-expression on long term potentiation (LTP) in the rTg4510 hippocampus before tangle formation. Mice were then killed at 4 months of age before neuronal loss manifests to assess whether Hsp27 was able to ameliorate the toxicity of aberrant tau intermediates over the 2-month period.

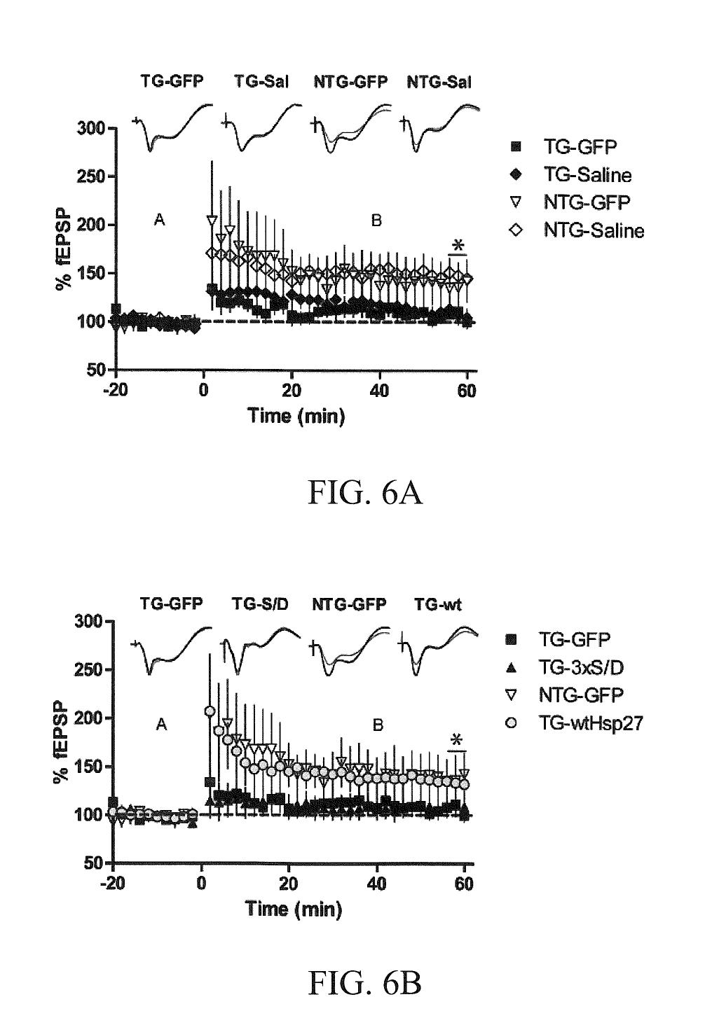

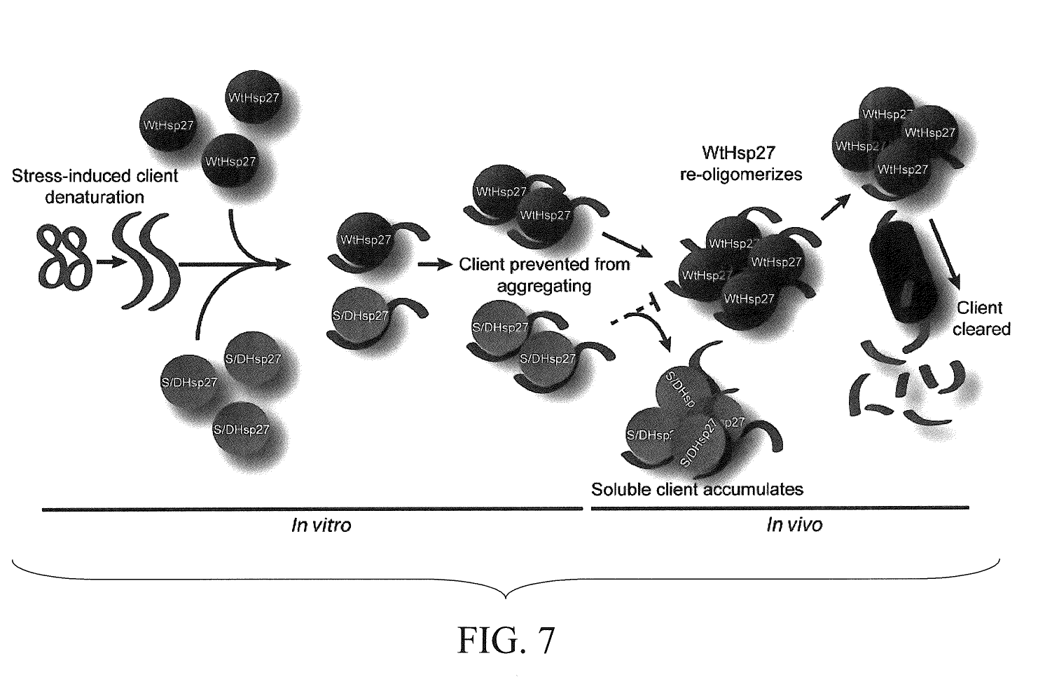

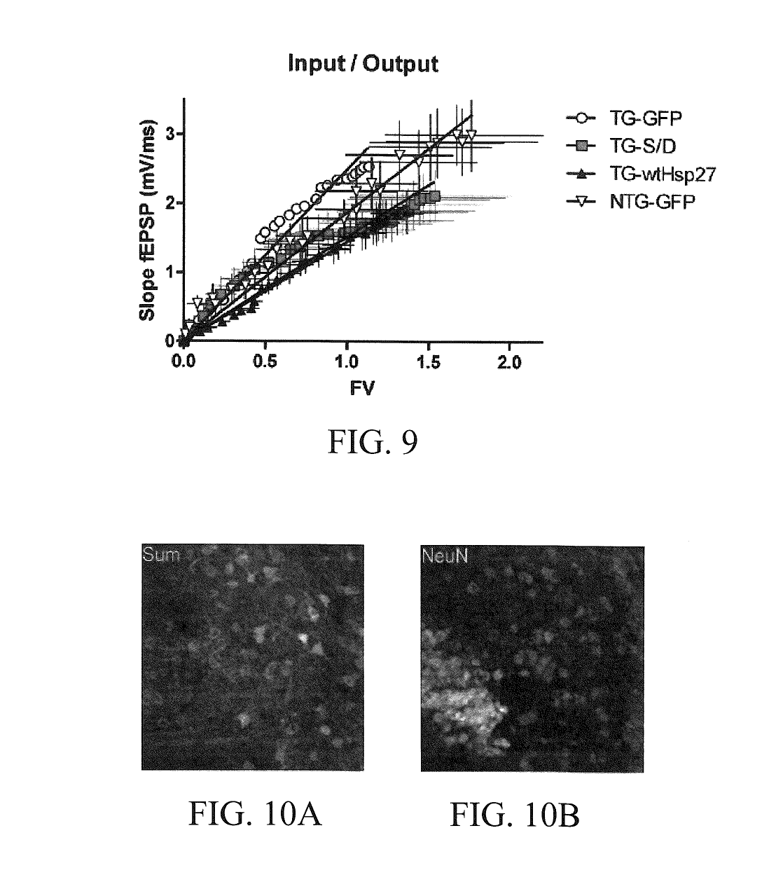

Hippocampal slices from each treatment group were stimulated by a high frequency theta burst (TBS) to assess LTP over a 60-minute period. Whether injected with saline or GFP-AAV9, there was no difference in the pattern of LTP in mice of the same genotype (FIG. 6A). The rTg4510 mice had significant reductions in LTP relative to NTG controls in either treatment (*p<0.001), showing LTP dysfunction in the rTg4510 model for the first time. Interestingly, LTP of hippocampal slices taken from rTg4510 mice transduced with wtHsp27-AAV9 was identical to NTG mice injected with GFP-AAV9. Western blot confirmed reductions in tau levels (FIGS. 8A-8B). Thus, wtHsp27 was able to functionally rescue hippocampal LTP deficits. Conversely, LTP deficits were equivalent in rTg4510 mice receiving 3×S/D Hsp27-AAV9 and GFP-AAV9 (FIG. 6B). Input/output analysis was done by plotting the fEPSP slopes to pre-synaptic fiber volley amplitude, and showed no significant differences between rTg4510 mice receiving either Hsp27 variant compared to those receiving GFP (FIG. 9). This analysis indicates that the functional rescue of LTP by wtHsp27 over-expression in rTg4510 mice was not due to changes in baseline synaptic transmission nor was viral induction generally altering pre-synaptic activity. These studies demonstrated that for Hsp27 to facilitate clearance of abnormal tau intermediates, it must be functionally intact with regard to phosphorylation dynamics. Moreover, since the LTP analyses were performed on 4 month-old rTg4510 hippocampal tissues, we can conclude that tau accumulation is sufficient to disrupt hippocampal function without killing neurons, since these mice do not have neuronal loss until after 5 months of age.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

U.S. Pat. No. 4,179,337
U.S. Pat. No. 5,753,502
U.S. Pat. No. 6,040,172
U.S. Pat. No. 6,066,726
U.S. Published Application No. 20040093630
Altschul, S. F. et al. (1990) “Basic Local Alignment Search Tool” J. Mol. Biol. 215:403-410.
Altschul, S. F. et al. (1997) “Gapped BLAST and PSI-BLAST: A New Generation of Protein Database Search Programs” Nucl. Acids Res. 25:3389-3402.
Beltz, G. A., Jacobs, K. A., Eickbush, T. H., Cherbas, P. T., Kafatos, F. C. (1983) “Isolation of multigene families and determination of homologies by filter hybridization methods” Methods of Enzymology, R. Wu, L. Grossman and K. Moldave [eds.] Academic Press, New York 100:266-285.
Feigner, P. L., T. R. Gadek, M. Holm, R. Roman, H. W. Chan, M. Wenz, J. P. Northrop, G. M. Ringold, M. Danielsen (1987) “Lipofection: a highly efficient, lipid-mediated DNA-transfection procedure” Proc Natl Acad Sci U.S.A. 84(21):7413-7417.
Karlin S., Altschul, S. F. (1990) “Methods for Assessing the Statistical Significance of Molecular Sequence Features by Using General Scoring Schemes” Proc. Natl. Acad. Sci. USA 87:2264-2268.
Karlin S., Altschul, S. F. (1993) “Applications and Statistics for Multiple High-Scoring Segments in Molecular Sequences” Proc. Natl. Acad. Sci. USA 90:5873-5877.
Maniatis, T., Fritsch, E. F., Sambrook, J. (1982) Molecular Cloning: A Laboratory Manual, Cold Spring Harbor Laboratory, Cold Spring Harbor, N.Y.
Barghorn S, Davies P, Mandelkow E (2004) Tau paired helical filaments from Alzheimer's disease brain and assembled in vitro are based on beta-structure in the core domain. Biochemistry 43:1694-1703.
Ben-Zvi A, Miller E A, Morimoto R I (2009) Collapse of proteostasis represents an early molecular event in Caenorhabditis elegans aging. Proc Natl Acad Sci USA 106:14914-14919.
Berrow N S, Bussow K, Coutard B, Diprose J, Ekberg M, Folkers G E, Levy N, Lieu V, Owens R J, Peleg Y, Pinaglia C, Quevillon-Cheruel S, Salim L, Scheich C, Vincentelli R, Busso D (2006) Recombinant protein expression and solubility screening in Escherichia coli: a comparative study. Acta Crystallogr D Biol Crystallogr 62:1218-1226.
Bobo R H, Laske D W, Akbasak A, Morrison P F, Dedrick R L, Oldfield E H (1994) Convection-enhanced delivery of macromolecules in the brain. Proc Natl Acad Sci USA 91:2076-2080.
Bolte S, Cordelieres F P (2006) A guided tour into subcellular colocalization analysis in light microscopy. J Microsc 224:213-232.
Carty N C, Nash K, Lee D, Mercer M, Gottschall P E, Meyers C, Muzyczka N, Gordon M N, Morgan D (2008) Adeno-associated viral (AAV) serotype 5 vector mediated gene delivery of endothelin-converting enzyme reduces Abeta deposits in APP+PS1 transgenic mice. Mol Ther 16:1580-1586.
Cearley C N, Vandenberghe L H, Parente M K, Carnish E R, Wilson J M, Wolfe J H (2008) Expanded repertoire of AAV vector serotypes mediate unique patterns of transduction in mouse brain. Mol Ther 16:1710-1718.
Cohen E, Du D, Joyce D, Kapernick E A, Volovik Y, Kelly J W, Dillin A (2010) Temporal requirements of insulin/IGF-1 signaling for proteotoxicity protection. Aging Cell 9:126-134.
Collombet J M, Masqueliez C, Four E, Burckhart M F, Bernabe D, Baubichon D, Lallement G (2006) Early reduction of NeuN antigenicity induced by soman poisoning in mice can be used to predict delayed neuronal degeneration in the hippocampus. Neurosci Lett 398:337-342.
Dickey C, Kraft C, Jinwal U, Koren J, Johnson A, Anderson L, Lebson L, Lee D, Dickson D, de Silva R, Binder L I, Morgan D, Lewis J (2009) Aging analysis reveals slowed tau turnover and enhanced stress response in a mouse model of tauopathy. Am J Pathol 174:228-238.
Frost B, Diamond M I (2009) Prion-like mechanisms in neurodegenerative diseases. Nat Rev Neurosci.
Fu H, Muenzer J, Samulski R J, Breese G, Sifford J, Zeng X, McCarty D M (2003) Self-complementary adeno-associated virus serotype 2 vector: global distribution and broad dispersion of AAV-mediated transgene expression in mouse brain. Mol Ther 8:911-917.
Gordon M N, Schreier W A, Ou X, Holcomb L A, Morgan D G (1997) Exaggerated astrocyte reactivity after nigrostriatal deafferentation in the aged rat. J Comp Neurol 388:106-119.
Gordon M N, Holcomb L A, Jantzen P T, DiCarlo G. Wilcock D, Boyett K W, Connor K, Melachrino J, O'Callaghan J P, Morgan D (2002) Time course of the development of Alzheimer-like pathology in the doubly transgenic PS1+APP mouse. Exp Neurol 173:183-195.
Hadaczek P, Kohutnicka M, Krauze M T, Bringas J, Pivirotto P, Cunningham J, Bankiewicz K (2006) Convection-enhanced delivery of adeno-associated virus type 2 (AAV2) into the striatum and transport of AAV2 within monkey brain. Hum Gene Ther 17:291-302.
Haley D A, Bova M P, Huang Q L, McHaourab H S, Stewart P L (2000) Small heat-shock protein structures reveal a continuum from symmetric to variable assemblies. J Mol Biol 298:261-272.
Hensley K, Floyd R A, Zheng N Y, Nael R, Robinson K A, Nguyen X, Pye Q N, Stewart C A, Geddes J, Markesbery W R, Patel E, Johnson G V, Bing G (1999) p38 kinase is activated in the Alzheimer's disease brain. J Neurochem 72:2053-2058.
Hill S E, Robinson J, Matthews G, Muschol M (2009) Amyloid protofibrils of lysozyme nucleate and grow via oligomer fusion. Biophys J 96:3781-3790.
Huot J, Roy G, Lambert H, Chretien P, Landry J (1991) Increased survival after treatments with anticancer agents of Chinese hamster cells expressing the human Mr 27,000 heat shock protein. Cancer Res 51:5245-5252.
Jakob U, Gaestel M, Engel K, Buchner J (1993) Small heat shock proteins are molecular chaperones. The Journal of biological chemistry 268:1517-1520.
Kienzler F, Norwood B A, Sloviter R S (2009) Hippocampal injury, atrophy, synaptic reorganization, and epileptogenesis after perforant pathway stimulation-induced status epilepticus in the mouse. J Comp Neurol 515:181-196.
Koren J, 3rd, Jinwal U K, Lee D C, Jones J R, Shults C L, Johnson A G, Anderson L J, Dickey C A (2009) Chaperone signalling complexes in Alzheimer's disease. J Cell Mol Med 13:619-630.
Landry J, Lambert H, Zhou M, Lavoie J N, Hickey E, Weber L A, Anderson C W (1992) Human HSP27 is phosphorylated at serines 78 and 82 by heat shock and mitogen-activated kinases that recognize the same amino acid motif as S6 kinase II. The Journal of biological chemistry 267:794-803.
Lelj-Garolla B, Mauk A G (2005) Self-association of a small heat shock protein. J Mol Biol 345:631-642.
Lelj-Garolla B, Mauk A G (2006) Self-association and chaperone activity of Hsp27 are thermally activated. The Journal of biological chemistry 281:8169-8174.
Levites Y, Jansen K, Smithson L A, Dakin R, Holloway V M, Das P, Golde T E (2006) Intracranial adeno-associated virus-mediated delivery of anti-pan amyloid beta, amyloid beta40, and amyloid beta42 single-chain variable fragments attenuates plaque pathology in amyloid precursor protein mice. J Neurosci 26:11923-11928.
McPhail L T, McBride C B, McGraw J, Steeves J D, Tetzlaff W (2004) Axotomy abolishes NeuN expression in facial but not rubrospinal neurons. Exp Neurol 185:182-190.
Miller G (2009) Neurodegeneration. Could they all be prion diseases? Science 326:1337-1339.
Morcol T, Subramanian A (1999) A red-dot-blot protein assay technique ii nanogram range. Anal Biochem 270:75-82.
Perrin V, Regulier E, Abbas-Terki T, Hassig R, Brouillet E, Aebischer P, Luthi-Carter R, Deglon N (2007) Neuroprotection by Hsp104 and Hsp27 in lentiviral-based rat models of Huntington's disease. Mol Ther 15:903-911.
Ramsden M, Kotilinek L, Forster C, Paulson J, McGowan E, SantaCruz K, Guimaraes A, Yue M, Lewis J, Carlson G, Hutton M, Ashe K H (2005) Age-dependent neurofibrillary tangle formation, neuron loss, and memory impairment in a mouse model of human tauopathy (P301L). J Neurosci 25:10637-10647.
Rasband M N (2009) Converging on the origins of axonal ion channel clustering. PLoS Genet. 5:e1000340.
Renkawek K, Bosman G J, de Jong W W (1994) Expression of small heat-shock protein hsp 27 in reactive gliosis in Alzheimer disease and other types of dementia. Acta Neuropathol 87:511-519.
Renkawek K, Stege G J, Bosman G J (1999) Dementia, gliosis and expression of the small heat shock proteins hsp27 and alpha B-crystallin in Parkinson's disease. Neuroreport 10:2273-2276.
Reynolds M R, Berry R W, Binder L I (2005) Site-specific nitration differentially influences tau assembly in vitro. Biochemistry 44:13997-14009.
Rocher A B, Crimins J L, Amatrudo J M, Kinson M S, Todd-Brown M A, Lewis J, Luebke J I (2009) Structural and functional changes in tau mutant mice neurons are not linked to the presence of NFTs. Exp Neurol.
Sanbe A, Yamauchi J, Miyamoto Y, Fujiwara Y, Murabe M, Tanoue A (2007) Interruption of CryAB-amyloid oligomer formation by HSP22. The Journal of biological chemistry 282:555-563.
Santacruz K, Lewis J, Spires T, Paulson J, Kotilinek L. Ingelsson M, Guimaraes A, DeTure M, Ramsden M, McGowan E, Forster C, Yue M, Orne J, Janus C, Mariash A, Kuskowski M, Hyman B, Hutton M, Ashe K H (2005) Tau suppression in a neurodegenerative mouse model improves memory function. Science 309:476-481.
Shimura H, Miura-Shimura Y, Kosik K S (2004) Binding of tau to heat shock protein 27 leads to decreased concentration of hyperphosphorylated tau and enhanced cell survival. The Journal of biological chemistry 279:17957-17962.
Stokoe D, Engel K, Campbell D G, Cohen P, Gaestel M (1992) Identification of MAPKAP kinase 2 as a major enzyme responsible for the phosphorylation of the small mammalian heat shock proteins. FEBS Lett 313:307-313.
Unal-Cevik I, Kilinc M, Gursoy-Ozdemir Y, Gurer G, Dalkara T (2004) Loss of NeuN immunoreactivity after cerebral ischemia does not indicate neuronal cell loss: a cautionary note. Brain Res 1015:169-174.
Wyttenbach A, Sauvageot O, Carmichael J, Diaz-Latoud C, Arrigo A P, Rubinsztein D C (2002) Heat shock protein 27 prevents cellular polyglutamine toxicity and suppresses the increase of reactive oxygen species caused by huntingtin. Hum Mol Genet 11:1137-1151.

1. A method for treating or preventing a neurodegenerative disease or condition associated with aggregation of the microtubule-associated protein tau in a person or animal, said method comprising administering or delivering an effective amount of a heat shock protein 27 (Hsp 27), or a biologically-active fragment thereof, to a person or animal in need to treatment.

2. The method according to claim 1, wherein said method further comprises identifying a person or animal in need of treatment.

3. The method according to claim 1, wherein said disease or condition is characterized by the presence of neurofibrillary tangles of protein tau and/or hyperphosphorylation of protein tau in a neuronal or glial cell.

4. The method according to claim 1, wherein said disease or condition is Alzheimer's disease, gangliogliomas, gangliocytomas, argyrophilic grain dementia, corticobasal degeneration, dementia pugilistica, frontotemporal dementia with parkinsonism linked to chromosome17, Pick's disease, Hallervorden-Spatz disease, myotonic dystrophy, Niemann-Pick disease (type C), Parkinsonism-dementia complex of Guam, postencephalitic parkinsonism, prion diseases, progressive subcortical gliosis, or progressive supranuclear palsy.

5. The method according to claim 1, wherein said Hsp 27, or a biologically active fragment thereof, is administered orally, nasally, rectally, parenterally, subcutaneously, intramuscularly, intraspinal, intracranially, or intravenously.

6. The method according to claim 1, wherein said Hsp 27 is administered to the person or animal as a polynucleotide encoding said Hsp 27, or said biologically active fragment thereof.

7. The method according to claim 6, wherein said polynucleotide is provided in an expression construct.

8. The method according to claim 6, wherein said Hsp 27 is a human Hsp 27.

9. The method according to claim 6, wherein said polynucleotide is injected directly into neural tissue and/or cells of the person or animal.

10. The method according to claim 9, wherein said neural tissue is hippocampal tissue.

11. The method according to claim 7, wherein said expression construct comprises a promoter that provides for expression of said polynucleotide in neurons or glial cells.

12. The method according to claim 7, wherein said expression construct is an adeno-associated viral construct.

13. The method according to claim 1, wherein said method further comprises administering other drugs and/or therapeutics used in treating neurodegenerative diseases and/or conditions.

14. The method according to claim 13, wherein said other drugs and/or therapeutics are administered prior to or at the same time or subsequent to administration of said Hsp 27, or said biologically active fragment thereof.

15. The method according to claim 13, wherein said other drugs and/or therapeutics are one or more of donepezil, galantamine, rivastigmine, memantine, or L-dopa.

16. The method according to claim 1, wherein said Hsp 27, or said biologically active fragment thereof, is capable of being in a phosphorylated form and a non-phosphorylated form.

17. The method according to claim 1, wherein said Hsp 27, or said biologically active fragment thereof, comprises an attached group that enhances cellular uptake of said Hsp 27.

18. The method according to claim 1, wherein said Hsp 27, or said biologically active fragment thereof, is encapsulated in a liposome.

19. The method according to claim 1, wherein said Hsp 27, or said biologically active fragment thereof, comprises an attached polyethylene glycol group or comprises an attached lipophilic moiety that provides for improved cell membrane permeability.

20. (canceled)

21. A method for improving memory function in a person or animal, said method comprising administering or delivering an effective amount of a heat shock protein 27 (Hsp 27), or a biologically-active fragment thereof, to a person or animal in need to treatment; or
for decreasing the level of protein tau in a cell and/or preventing or reducing protein tau from aggregating in a cell, said method comprising contacting or providing or delivering to said cell an effective amount of an Hsp 27, or a biologically active fragment thereof; or
for treating and/or preventing hippocampal long-term potentiation decay or impairment in a person or animal, said method comprising administering or delivering an effective amount of a heat shock protein 27 (Hsp 27), or a biologically-active fragment thereof, to a person or animal in need to treatment.

22-76. (canceled)

77. A composition of matter comprising:
a) a composition comprising i) an Hsp 27, or a biologically active fragment thereof; and/or ii) a polynucleotide encoding an Hsp 27, or a biologically active fragment thereof; or
b) a packaged dosage formulation comprising at least one of i) an Hsp 27, or a biologically active fragment thereof; and/or ii) a polynucleotide encoding an Hsp 27, or a biologically active fragment thereof; in a pharmaceutically acceptable dosage in one or more packages, packets, or containers; or
c) a kit comprising in one or more containers i) an Hsp 27, or a biologically active fragment thereof, and/or ii) a polynucleotide encoding said Hsp 27, or said biologically active fragment thereof.

78-128. (canceled)
